(19) United States
(12) Patent Application Publication
(10) Pub. No.: US 2024/0366537 A1
(43) Pub. Date: Nov. 7, 2024

(54) THERAPEUTIC PATCH AND USES THEREOF

(71) Applicant: ORIPATCH AS, Kristiansand (NO)

(72) Inventors: Eva Cathrine Aardal SKUTHE, Kristiansand (NO); Jostein SKUTHE, Kristiansand (NO); Kjetil HENRIKSEN, Kristiansand (NO)

(21) Appl. No.: 18/577,558

(22) PCT Filed: Jul. 8, 2022
(86) PCT No.: PCT/EP2022/069101

§ 371 Date: Jan. 8, 2024

(30) Foreign Application Priority Data
Jul. 8, 2021 (GB) 2109849.6

(51) Int. Cl. A61K 31/167; A61K 9/00; A61K 9/14; A61K 31/416; A61K 47/32; A61K 47/36; A61K 47/38

(52) U.S. Cl. CPC A61K 31/167; A61K 9/006; A61K 9/143; A61K 31/416; A61K 47/32; A61K 47/36; A61K 47/38

(57) ABSTRACT

The present invention relates to therapeutic patches, particularly to soluble patches that adhere to mucosal membranes (e.g. in the oral cavity) to deliver analgesic (e.g. local anaesthetic) compounds (e.g. lidocaine) to sites of pain. In particular, the invention provides a mucoadhesive patch comprising a pharmaceutically acceptable soluble film and mesoporous microparticles loaded with an analgesic agent.

### FIELD OF THE INVENTION

The present invention relates to therapeutic patches, particularly to soluble patches that adhere to mucosal membranes (e.g. in the oral cavity) to deliver analgesic (e.g. local anaesthetic) compounds (e.g. lidocaine) to sites of pain. The therapeutic patch provided herein may also contain other therapeutically active agents, such as agents for treating disorders or conditions of, or trauma in, the oral cavity, e.g. anti-inflammatory agents, antiseptic agents and/or hyaluronic acid. The therapeutic patch finds utility in therapy, particularly in treating painful disorders or conditions of, or trauma in, of the oral cavity, such as aphthous stomatitis. Methods for producing the therapeutic patch and a packaged product containing the patch are also provided.

### BACKGROUND TO THE INVENTION

Aphthous stomatitis (also known as aphthous ulcers or canker sores) is a common condition affecting about 20% of the population. The condition is characterized by the repeated formation of benign and non-contagious mouth ulcers (aphthae). There are several forms of aphthous stomatitis including recurrent aphthous stomatitis (RAS), such as minor recurrent aphthous stomatitis (MiRAS) and major recurrent aphthous stomatitis (MaRAS), and herpetiform ulcers. Each episode may result in the formation of a plurality of ulcers and may therefore be characterised as multiple aphthous stomatitis. Typically, the ulcers occur periodically on the non-keratinizing epithelial surfaces in the mouth. However, some severe forms of the condition may also involve keratinizing epithelial surfaces.

Ulceration episodes commonly occur 3-6 times per year and while ulcers normally heal completely between attacks, individual ulcers often last about 7-10 days and can cause significant pain, which may result in problems with eating and drinking, speaking, movement of the mouth, playing mouth instruments etc. For severe forms of the condition the ulcers may be debilitating, leading to weight loss due to malnutrition.

Currently there is no cure for aphthous stomatitis and treatments focus on the management of pain and inflammation. However, commercially available treatments can be difficult to apply (e.g. analgesic gels), do not provide long-lasting effects and/or may not be suitable for both adults and children. The use of oral patches or adhesive tablets to deliver analgesics for pain relief associated with aphthous stomatitis has been proposed (e.g. WO 2009/013562 and US 2007/042027) but these have undesirable properties, such as being insoluble and therefore requiring the subject to remove the product after the analgesic effect has been achieved.

Thus, there is a need for improved products for delivering analgesics (e.g. topical anaesthetics) to the oral cavity, e.g. for the treatment (e.g. pain relief) of disorders of the oral cavity.

### SUMMARY OF THE INVENTION

In work leading up to the present invention, the inventors were approached by a patient suffering from problematic aphthous stomatitis who was unable to find effective products that could help to heal and relieve pain associated with the ulcers. The inventors initially developed an oral patch composed of lidocaine gel interposed between sugar-plates that could adhere to the oral mucosa and found that it was surprisingly effective at relieving pain from the ulcers for up to about 90 minutes.

Based on this initial success the inventors sought to modify and improve the initial prototype to provide a product with desirable and advantageous properties. In particular, the inventors sought to produce an oral patch that adheres to the oral mucosa and: is small, simple and convenient to apply to a site of pain (which therefore minimises the risk of user errors); provides a rapid onset of action (i.e. pain relief); causes no or minimal pain on application; is soluble (thereby avoiding the need to remove the patch, which can be inconvenient and could be painful); provides a sustained local effect with a well-defined treatment area; and gives a low effective dose (thereby minimising the risk of side effects, such as numbing of other areas which can result in bite injuries and throat/speech/swallowing problems).

The inventors hypothesised that this extensive list of characteristics could be satisfied by providing an oral soluble film loaded with a powdered form of lidocaine, which is a well-known active substance that is safe and effective for both adults and children. As shown in the Examples, representative successful embodiments of the invention were developed using mesoporous silica microparticles (MSMs) loaded with lidocaine hydrochloride adhered to an alginate-based film. The inventors have shown that these patches are capable of adhering to the oral mucosa and effecting sustained release of the lidocaine compound in amounts suitable to achieve effective and prolonged local pain relief. Moreover, the patches are soluble and therefore avoid the need actively to remove them from the site of action.

The therapeutic patch of the invention is expected to find utility not only in the treatment of aphthous stomatitis but as a generic therapy for painful disorders of the oral cavity, such as lesions (e.g. sores, ulcers, wounds etc.) caused by mechanical, chemical, radiation or idiopathic trauma, and as a pain relief in dentition, e.g. in tooth eruption.

Thus, in one aspect the invention provides a mucoadhesive patch comprising a pharmaceutically acceptable soluble film and mesoporous microparticles loaded with an analgesic (e.g. local or topical anaesthetic) agent.

In another aspect, the invention provides a mucoadhesive patch of the invention for use in therapy.

Alternatively viewed, the invention provides a method for treating and/or preventing a disease, condition or trauma (e.g. a disease, disorder, condition or trauma of the oral cavity) in a subject, which comprises the step of administering a mucoadhesive patch according to the invention to the subject (e.g. at a site of pain on a mucosal membrane, e.g. in the oral cavity).

From another perspective, the invention provides the use of a pharmaceutically acceptable soluble film and mesoporous microparticles loaded with an analgesic agent in the manufacture of a mucoadhesive patch for therapy.

In a further aspect, the invention provides a mucoadhesive patch of the invention for use in treating (e.g. relieving) and/or preventing pain, e.g. pain associated with or in proximity to a mucosal membrane, such as oral mucosa.

Alternatively viewed, the invention provides a method for treating (e.g. relieving) and/or preventing pain (e.g. pain associated with or in proximity to a mucosal membrane, such as oral mucosa) in a subject, which comprises the step of administering a mucoadhesive patch according to the invention to the subject (e.g. at or in proximity to the site of pain or expected pain, i.e. to a mucosal membrane, such as oral mucosa).

From another perspective, the invention provides the use of a pharmaceutically acceptable soluble film and mesoporous microparticles loaded with an analgesic agent in the manufacture of a mucoadhesive patch for treating (e.g. relieving) and/or preventing pain, e.g. pain associated with or in proximity to a mucosal membrane, such as oral mucosa.

In yet another aspect, the invention provides a mucoadhesive patch of the invention for use in treating disorders, conditions or trauma of the oral cavity, such as lesions (e.g. sores, ulcers, wounds etc.) of the oral mucosa, e.g. aphthous stomatitis.

Alternatively viewed, the invention provides a method for treating disorders of the oral cavity (e.g. lesions (e.g. sores, ulcers, wounds etc.) of the oral mucosa, such as aphthous stomatitis) in a subject, which comprises the step of administering a mucoadhesive patch according to the invention to the subject (e.g. at or in proximity to the site of the sore or lesion).

From another perspective, the invention provides the use of a pharmaceutically acceptable soluble film and mesoporous microparticles loaded with an analgesic agent in the manufacture of a mucoadhesive patch for treating disorders, conditions or trauma of the oral cavity, such as lesions (e.g. sores, ulcers, wounds etc.) of the oral mucosa, e.g. aphthous stomatitis.

In another embodiment, the invention provides a packaged product comprising the mucoadhesive patch of the invention.

In a further embodiment, the invention provides a method for producing a mucoadhesive patch of the invention comprising:
(a) providing: (i) a pharmaceutically acceptable soluble film; and (ii) mesoporous microparticles loaded with an analgesic agent;
(b)(i) adhering the mesoporous microparticles loaded with an analgesic agent to a surface of the film of (a)(i); or (ii) encapsulating the mesoporous microparticles loaded with an analgesic agent between the film of (a)(i) and a second pharmaceutically acceptable soluble film, optionally wherein the second film is the same as the film of (a)(i).

In a further embodiment, the invention provides a method for producing a mucoadhesive patch comprising:
(i) transferring a solution comprising: (a) alginate and optionally hyaluronic acid; and (b) mesoporous microparticles loaded with an analgesic agent, to a substrate or support (e.g. cast or mould) in an amount suitable to prepare a mucoadhesive pharmaceutically acceptable soluble film in which the mesoporous microparticles are distributed or dispersed within the film;
(ii) incubating the substrate or support under conditions to produce a mucoadhesive pharmaceutically acceptable soluble film, thereby producing the mucoadhesive patch; optionally
(iii) transferring a solution comprising hydroxypropyl methyl cellulose (HPMC) and optionally polyvinylpyrrolidone (PVP) to the surface of the mucoadhesive pharmaceutically acceptable soluble film produced in (ii) in an amount suitable to prepare a film comprising HPMC and optionally PVP adhered to the mucoadhesive pharmaceutically acceptable soluble film; and
(iv) incubating the product of (iii) under conditions to suitable for the solution comprising HPMC and optionally PVP to dry, thereby producing the mucoadhesive patch.

While not wishing to be bound by theory, it is also contemplated that a mucoadhesive patch as described herein without mesoporous microparticles loaded with an analgesic agent may also be effective at relieving pain associated with disorders of the oral cavity, such as lesions (e.g. sores, ulcers, wounds etc.) caused by mechanical, chemical, radiation or idiopathic trauma, and in dentition, e.g. in tooth eruption.

Thus, in a further embodiment, the invention provides a mucoadhesive patch comprising a pharmaceutically acceptable soluble film substantially as described herein (i.e. wherein the mucoadhesive patch does not contain mesoporous microparticles loaded with an analgesic agent). Accordingly, in some embodiments, the invention provides a mucoadhesive patch consisting of, or essentially consisting of, one or more pharmaceutically acceptable soluble films substantially as described herein. Insofar as the mucoadhesive patch essentially consists of one or more pharmaceutically acceptable soluble films substantially as described herein, it may also contain an additional therapeutic agent as defined herein, e.g. hyaluronic acid, a coating as described herein etc. In some embodiments, the mucoadhesive patch comprises a pharmaceutically acceptable soluble alginate film substantially as described herein.

Thus, all of the embodiments set forth in the detailed description below relating to the mucoadhesive patch, pharmaceutically acceptable soluble film (e.g. alginate film) and additional therapeutic agents may be used to describe a mucoadhesive patch that does not contain mesoporous microparticles loaded with an analgesic agent.

Thus, in one aspect the invention provides a mucoadhesive patch comprising two layers, wherein:
a) a first layer comprises a mucoadhesive pharmaceutically acceptable soluble film; and
b) a second layer comprises a non-mucoadhesive pharmaceutically acceptable soluble film,
wherein a surface of the second layer is available to contact a mucosal membrane when the first layer is adhered to a mucosal membrane.

Moreover, any mucoadhesive patch of the invention (i.e. including patches that do not contain mesoporous microparticles loaded with an analgesic agent) may be used in any of the therapeutic utilities described herein, e.g. in the oral cavity.

The invention also provides a method for producing a mucoadhesive patch or mucoadhesive pharmaceutically acceptable soluble film comprising:
(i) transferring a solution comprising alginate and optionally hyaluronic acid to a substrate or support (e.g. cast or mould) in an amount suitable to prepare a film; and
(ii) incubating the substrate or support at a temperature of about 45-60° C. (e.g. about 50-55° C.) for at least about 8 hours (e.g. about 8-48 hours or about 12-24 hours) to produce said film or patch,
wherein step (ii) is performed: (a) under conditions that minimise air turbulence at the surface of the substrate or support; and/or (b) at a high relative humidity.

### DETAILED DESCRIPTION

The term “patch” refers to refers to a piece of material that is used to cover a specific area. Thus, in the context of the present invention, a patch refers to a piece of material (a film or sheet of a substance) that is suitable to cover an area of mucosal membrane at, or in proximity to, a site of pain, e.g. a lesion (e.g. sore, ulcer, wound etc.) on a mucosal membrane. As discussed further below, a patch may be composed of more than one piece of material (more than one film or sheet). In particular, a patch may be composed of a plurality of layers of material (e.g. two or more layers, e.g. 2, 3, 4 or more layers). In some embodiments, each layer may be made of the same material. In some embodiments, two or more layers may be composed of different materials, e.g. the patch may comprise a mucoadhesive layer and a non-mucoadhesive layer each composed of a different substance.

The term “mucoadhesive” refers to a substance that is capable of adhering to mucosa, i.e. a mucosal membrane. Thus, “a mucoadhesive patch” refers to a patch that adheres to mucosa upon contact with the mucosa under conditions that are suitable for adhesion to occur, e.g. contact with the mucosa under constant pressure for a period of time, e.g. application of finger pressure to the patch on the mucosa for at least about 5 seconds, e.g. about 6, 7, 8, 9, 10 or more seconds.

Accordingly, a mucoadhesive patch refers to a patch that adheres to mucosa (e.g. oral mucosa) in one place and does not detach and/or move substantially from the point of contact with the mucosa (following successful adhesion), i.e. does not detach and/or move from the point of contact without the application of substantive force, e.g. the minimum pull-off force as defined below. For instance, a mucoadhesive patch may cover at least 50%, e.g. at least 60, 70, 80 or 90%, of the area of mucosa to which it is initially contacted and adhered for substantially all of the duration of its contact with the mucosa, i.e. until it is substantially dissolved or removed. It will be understood that the area covered by the patch may decrease as the patch dissolves. Accordingly, a mucoadhesive patch may cover at least 50%, e.g. at least 60, 70, 80 or 90%, of the area of mucosa to which it is initially contacted and adhered until it is substantially dissolved, e.g. until the surface area of the patch is reduced by about 30% or more, e.g. about 40% or 50% or more, relative to the surface area of the patch prior to contact with the mucosa.

Thus, alternatively viewed, a mucoadhesive patch may not move more than about 20 mm, e.g. about 15 mm or 10 mm, from the area of mucosa to which it is initially contacted and adhered for substantially all of the duration of its contact with the mucosa, i.e. until it is substantially dissolved (e.g. until the surface area of the patch is reduced by about 30% or more, e.g. about 40% or 50% or more, relative to the surface area of the patch prior to contact with the mucosa) or removed. Movement of the patch may be determined using any fixed point of the patch, e.g. the centre of the patch.

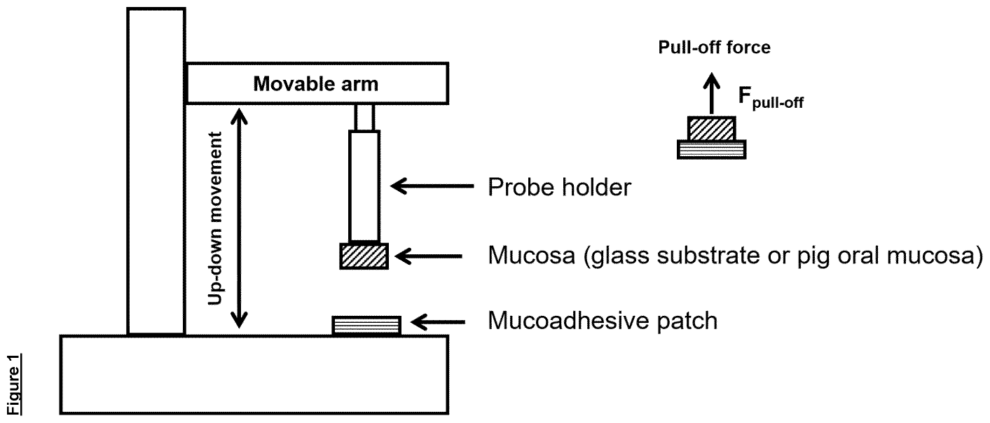

As shown in the Examples, the mucoadhesive properties of a patch may be evaluated by measuring the adhesivity of the patch using texture analyser equipment and a glass slide. A glass slide is a hydrophilic substrate that has been shown to be an appropriate model surface of hydrophilic surface mucosa, i.e. it is an appropriate mimic of oral mucosa. The principle of mucoadhesion measurement is shown in the schematic illustration in FIG. 1. The parameters measured are the pull-off force (N) and the work of adhesion (Ns).

In a representative example, a glass substrate (e.g. a 10×10 mm glass slide) is contacted with the patch (having a greater surface area than the glass substrate, i.e. an overlapping surface area, e.g. at least 110%, such as at least about 120%, 130% or 140%, of the surface area of the glass substrate, e.g. about 110%-200% of the surface area of the glass substrate) which has been wet with saliva or an equivalent solution (e.g. saliva-mimicking solution such as phosphate-buffered saline (about pH 6-8, e.g. about pH 7, such as about pH 7.4), e.g. about 10-30 μL, such as about 15 μL) until an appropriate contact force is reached, e.g. about 20 g-400 g, such as about 50 g-380 g, 100 g-360 g, 150 g-340 g, 200 g-320 g, e.g. about 300 g, and maintained for about 10 seconds, e.g. 5-20 seconds. The forced required to detach the patch from the glass substrate is then measured.

Thus, in some embodiments, the mucoadhesive patch (e.g. a soluble film) has an average pull-off force of at least about 2.0N, e.g. at least about 2.5N, 3.0N or 3.5N, such as about 4.0N or more, as measured using the method described above and in more detail in the Examples (e.g. wherein the force is an average of about 5 measurements). Thus, in some embodiments, the mucoadhesive patch that has been wet with a saliva-mimicking liquid, e.g. 15 μL of PBS (pH 7.4), has an average pull-off force of at least about 2.0N as measured using a glass substrate with a surface area of 1 cm². In some embodiments, the mucoadhesive patch has an average pull-off force of less about 15N, e.g. less than about 12N or 10N, e.g. the pull-off force is about 2.0-15N, 2.5-12N or 3.0-10N.

In some embodiments, the mucoadhesive patch has an average work of adhesion of at least about 0.05 Ns, e.g. at least about 0.10 Ns, 0.12 Ns, 0.14 Ns, 0.16 Ns, 0.18 Ns or 0.20 Ns, such as about 0.25 Ns or more, as measured using the method described above and in more detail in the Examples (e.g. wherein the force is an average of about 5 measurements). Thus, in some embodiments, the mucoadhesive patch that has been wet with a saliva-mimicking liquid, e.g. 15 μL of PBS (pH 7.4), has an average work of adhesion of at least about 0.05 Ns as measured using a glass substrate with a surface area of 1 cm². In some embodiments, the mucoadhesive patch has an average work of adhesion of less than about 5.0 Ns, e.g. less than about 4.0 Ns, 3.0 Ns or 2.0 Ns, e.g. the work of adhesion is about 0.05-5.0 Ns, 0.10-4.0 Ns, 0.12-3.0 Ns, 0.14-2.5 Ns or 0.16-2.0 Ns. In some embodiments, the mucoadhesive patch has an average work of adhesion of about 0.50-5.0 Ns, 0.55-4.0 Ns, 0.60-3.0 Ns, 0.65-2.5 Ns or 0.70-2.0 Ns

As noted above, the mucoadhesive patch of the invention finds particular utility in the treatment of disorders, conditions or trauma of the oral cavity and in the treatment and/or prevention of pain in the oral cavity, i.e. at local sites within the oral cavity. Thus, in some embodiments, the mucoadhesive patch of the invention is an oral or buccal mucoadhesive patch, i.e. it adheres to mucosa in the oral cavity, i.e. oral mucosa.

Thus, a “mucoadhesive patch” as defined herein does not include preparations (e.g. films, tablets, lozenges etc.) that move about on a mucosal membrane (e.g. in the oral cavity) rather than adhering in one place. Nor does it include preparations that do not hold together as a single item when held in the oral cavity such as preparations of powder, liquid, paste, viscous liquid gel, or a tablet or troche that crumbles into a powder or paste when chewed or placed in saliva.

The terms “mucous membrane”, “mucosal membrane” and “mucosa” are used interchangeably herein to refer to membranes that line various cavities in the body including the mouth, eyes, ears, nose, vagina, the urethral opening and the anus.

The oral mucosa refers to the mucous membrane lining the inside of the mouth and comprises the lining mucosa, masticatory mucosa and specialized mucosa. The lining mucosa comprises non-keratinized stratified squamous epithelium and makes up the buccal mucosa (the inside lining of the cheeks and floor of the mouth), the labial mucosa (the inside lining of the lips) and the alveolar mucosa (the lining between the buccal and labial mucosae). The masticatory mucosa comprises keratinized stratified squamous epithelium found on the dorsum of the tongue, hard palate and attached gingiva (gums). The specialized mucosa comprises the regions of the taste buds on lingual papillae on the dorsal surface of the tongue.

The mucoadhesive patch may be contacted with and adhered to any part of the oral mucosa. In some embodiments, the mucoadhesive patch may be contacted with and adhered to the lining mucosa (particularly the labial and/or buccal mucosa) and/or masticatory mucosa. It will be understood that the patch may be provided in any size or shape suitable for use in the oral cavity and therefore a single patch may be contacted with, and adhered to, more than part of the oral mucosa, i.e. it may cover an area comprising different types of oral mucosa, e.g. the inside lining of the cheeks and lips or the inside lining of the cheeks and gingiva etc.

In some embodiments, the mucoadhesive patch of the invention comprises a pharmaceutically acceptable soluble film and mesoporous microparticles loaded with an analgesic agent. It will be evident from the disclosures below that these components may be selected and arranged (i.e. configured) in numerous ways to achieve the desired functional properties of the patch as described herein (e.g. to achieve pain relief at a site of pain on a mucosal membrane). In particular, the components of the patch must be selected and arranged such that the patch is suitable for local administration of an analgesic (e.g. topical anaesthetic) agent to a mucosal membrane and/or suitable for achieving pain relief at a site of pain on a mucosal membrane. Accordingly, the components of the patch must be selected and arranged such that upon contact and adhesion of the patch to a mucosal membrane, the analgesic (e.g. topical anaesthetic) agent is released from the patch in an effective amount, i.e. an amount suitable to achieve pain relief. The components of the patch also must be selected and arranged such that the patch is soluble, e.g. the film components of the patch dissolve following adhesion to the mucosal membrane (e.g. oral mucosa). It will be understood that in embodiments where the patch contains mesoporous microparticles, these will not dissolve and thus a soluble mucoadhesive patch refers to the solubility of the soluble components of the patch, i.e. the film components.

A pharmaceutically acceptable soluble film or sheet refers to a thin layer of a substance or material that is capable of dissolving on contact with a mucosal membrane (e.g. oral mucosa). The film may also be mucoadhesive as defined above, i.e. a pharmaceutically acceptable soluble and mucoadhesive film. However, as noted above, the mucoadhesive patch of the invention may comprise a plurality of layers (e.g. films) and it will be evident that only the layer (film or sheet) that is contacted with the mucosa must have mucoadhesive properties, i.e. to enable the patch to adhere to the mucosa, as defined above. The film must also be flexible enough to be applied to an uneven, e.g. curved, surface such that it is suitable for adhesion to a mucosal membrane, e.g. oral mucosa. Any suitable material may be used to provide the film of the mucoadhesive patch of the invention.

The terms “pharmaceutically acceptable” and “physiologically acceptable” are used interchangeably herein to refer to materials or substances that must be compatible with other ingredients in the patch (e.g. the analgesic agent, mesoporous particles, additional therapeutic agents) as well as physiologically acceptable to the recipient. Furthermore, the material preferably is also sterile and pyrogen free.

A “soluble film or sheet” may be comprised of any pharmaceutically acceptable material that is capable of forming a film or sheet that is soluble in water and/or an aqueous solution, such as saliva or an equivalent thereof (e.g. phosphate-buffered saline). As shown in the Examples, the solubility of a film (e.g. a patch of the invention) may be evaluated by measuring the time taken for the film or patch (of defined size) to dissolve completely in an aqueous solution under controlled conditions.

In a representative example, a soluble film (e.g. 20×10 mm) is submerged in 100 mL phosphate-buffered saline (e.g. about pH 6-8, e.g. about pH 7) at room temperature (e.g. 20-25° C.), e.g. with gentle stirring (e.g. 10-100 rpm, such as about 50 rpm) until the film is no longer visible.

Thus, in some embodiments, a 20×10 mm piece of the soluble film used in the mucoadhesive patch of the invention or the mucoadhesive patch itself completely dissolves in 100 mL phosphate-buffered saline (e.g. about pH 6-8, e.g. about pH 7, such as about pH 7.4) at room temperature (e.g. 20-25° C.) in about 2 hours or less, e.g. 90, 75, 60, 45, 40, 35, 30, 25 minutes or less, such as about 10-90, 15-75 or 20-60 minutes.

Thus, in some embodiments, a mucoadhesive patch of the invention having a surface area of about 200 mm² dissolves completely in 100 mL phosphate-buffered saline, pH 7.4 at room temperature in about 30-90 minutes.

A soluble film or sheet for use in the mucoadhesive patch of the invention may be of any thickness suitable to achieve the required properties disclosed herein, e.g. solubility. In this respect, it will be understood that the thickness of the film may be altered to modulate the time needed for the patch to dissolve on the mucosa (e.g. oral mucosa). This may be particularly advantageous when the patch is used to treat very painful lesions, e.g. ulcers or sores, as the patch may function as a barrier to substances that may irritate the lesion, e.g. acidic foods. In a representative embodiment, a film or sheet in the patch (e.g. each sheet in the patch) may have a thickness of about 50-1000 μm, such as about 75-900 μm, 100-800 μm, 125-700 μm or 150-600 μm. Thus, in some embodiments, the patch may have a thickness of about 50-1000 μm, such as about 100-800 μm, about 150-700 μm or about 200-500 μm.

Pharmaceutically acceptable materials capable of forming a soluble (and mucoadhesive) film include water soluble polymers, such as alginate, cellulose and polyacrylic acid. Thus, in some embodiments, the pharmaceutically acceptable soluble film comprises alginate or cellulose or polyacrylic acid, or a combination thereof. In a preferred embodiment, the pharmaceutically acceptable soluble film comprises alginate. Thus, in some embodiments, the mucoadhesive pharmaceutically acceptable soluble film comprises alginate.

Any alginate capable of producing a film with the required solubility and mucoadhesive properties defined above may be used in the pharmaceutically acceptable soluble film. In some embodiments, the alginate comprises 25-45% (e.g. 30-40%) guluronate and 55-75% (e.g. 60-70%) mannuronate. Suitable pharmaceutical grade formulations of alginate are commercially available (e.g. Manucol® LKX, Protanal® CR 8133, Protanal® CR 8233) and may be used in the present invention. Protanal® CR 8133, Manucol® LKX and Protanal® CR 8233 represent low viscosity, medium viscosity and high viscosity alginates, respectively, wherein the viscosity is determined for a 1-2% w/v solution at about 20° C. In some preferred embodiments, the alginate used in the pharmaceutically acceptable soluble film is a medium viscosity alginate, a high viscosity alginate or a combination thereof.

Similarly, any cellulose polymer capable of producing a film with the required solubility and mucoadhesive properties defined above may be used in the pharmaceutically acceptable soluble film. In some embodiments, the cellulose polymer comprises hydroxypropyl methylcellulose (HPMC), hydroxypropyl cellulose (HPC), hydroxyethyl cellulose (HEC), sodium carboxymethyl cellulose (CMC) or a combination thereof. In some embodiments, a pharmaceutically acceptable soluble film comprising a cellulose polymer (e.g. HPMC) is a non-mucoadhesive film.

In a preferred embodiment, the pharmaceutically acceptable soluble film comprises an alginate polymer comprising 30-40% guluronate and 60-70% mannuronate (e.g. Manucol® LKX, Protanal® CR 8133, Protanal® CR 8233), preferably wherein a 1% w/v solution of the alginate (e.g. a medium viscosity alginate, such as Manucol® LKX) has a viscosity of about 60-170 mPa·s (e.g. at about 20° C.), preferably about 80-130 mPa·s (e.g. at about 20° C.). In some embodiments, a 2% w/v solution of the alginate polymer (e.g. a low viscosity alginate, such as an alginate comprising 30-40% guluronate and 60-70% mannuronate, e.g. Protanal® CR 8133) used to produce the pharmaceutically acceptable soluble film has a viscosity of about 100-300 mPa·s (e.g. at about 20° C.), preferably about 120-250 mPa·s (e.g. at about 20° C.). In some embodiments, a 1.25% w/v solution of the alginate polymer (e.g. a high viscosity alginate, such as an alginate comprising 30-40% guluronate and 60-70% mannuronate, e.g. Protanal® CR 8233) used to produce the pharmaceutically acceptable soluble film has a viscosity of about 600-900 mPa·s (e.g. at about 20° C.), preferably about 650-850 mPa·s (e.g. at about 20° C.).

The viscosity of a solution (e.g. a polymer solution, such as an alginate solution) may be determined using any suitable means known in the art, e.g. using a viscometer such as a capillary kinematic viscometer or a rotational viscometer.

The pharmaceutically acceptable soluble film may be prepared using any suitable means. In a representative example, and as described in the Examples below, the film may be produced by preparing a solution of the polymer that is subsequently transferred to a substrate or support, e.g. cast or mould (by any suitable means known in the art, e.g. pouring, spraying etc.), and allowed to set (e.g. cure or dry) in order to prepare the film. It will be evident that the film may be prepared using any suitable means. For instance, the film may be prepared in casts or moulds to allow direct assembly of the mucoadhesive patch of the invention. Alternatively, a large film or sheet may be prepared and subsequently cut to into smaller pieces suitable for use in the mucoadhesive patch of the invention or subsequent to the assembly of the other components of the patch, i.e. the loaded mesoporous microparticles and optionally one or more additional layers, coatings etc.

As shown in the Examples below, it has been determined that the method used to dry or cure the film may impact on the uniformity of the film and thus the consistency of patches produced from the film. In particular, specific drying conditions may function to improve the uniformity of the film and the weight of the patches produced therefrom.

In particular, it has been surprisingly found that incubating the film at temperatures of at least 45° C. (e.g. about 45-60° C. such about 50-55° C., e.g. about 50° C.) for at least about 8 hours (e.g. about 8-48 hours or about 12-24 hours) functions to improve the homogeneity of the film.

Moreover, incubating the film at a high relative humidity also improves the homogeneity of the film.

The term “relative humidity” refers to the ratio of the partial pressure of water vapour to the equilibrium vapour pressure of water at a given temperature. A high relative humidity typically refers to conditions in which the relative humidity is at least about 40%.

Any suitable means for increasing the relative humidity of the environment in which the film is dried (e.g. drying cabinet) may be used in the methods disclosed herein and is within the purview of the skilled person. For instance, incubating the film (i.e. the support or substrate on which the film is produced) in proximity to an open container comprising a salt solution (e.g. NaCl) may increase the relative humidity and thereby improve the homogeneity of the film.

Thus, in some embodiments, the step of incubating the substrate or support on which the film is drying is performed at a relative humidity of at least about 40%, preferably at least about 45%, 50%, 55%, 60%, 65% or 70%.

It also has been determined that the homogeneity of the film may be improved by drying the film under conditions in which the air turbulence at the surface of the substrate or support on which the film is drying is minimised.

“Air turbulence” refers to an irregular motion of the air resulting from eddies and vertical currents. In the context of the present invention, air turbulence may be the result of the movement of air in a drying cabinet required to maintain constant temperature and/or humidity conditions. Any suitable means for reducing or minimising air turbulence at the surface of the support or substrate may be used in the methods disclosed herein and is within the purview of the skilled person. For instance, air turbulence may be minimised by placing the substrate in a container comprising one or a plurality of perforations, e.g. a container with a perforated lid.

Accordingly, in some embodiments the invention provides a method for producing a mucoadhesive patch or mucoadhesive pharmaceutically acceptable soluble film comprising:
(i) transferring a solution comprising alginate and optionally hyaluronic acid to a substrate or support (e.g. cast or mould) in an amount suitable to prepare a film; and
(ii) incubating the substrate or support at a temperature of about 45-60° C. (e.g. about 50-55° C.) for at least about 8 hours (e.g. about 8-48 hours or about 12-24 hours) to produce said film or patch,
wherein step (ii) is performed: (a) under conditions that minimise air turbulence at the surface of the substrate or support; and/or (b) at a high relative humidity.

Any suitable concentration of polymer, e.g. alginate, may be used to prepare the “film solution” for the pharmaceutically acceptable soluble film and is within the purview of the skilled person. In some embodiments, the concentration of polymer, e.g. alginate, in the film solution is about 0.5-5.0% w/w, e.g. about 1.0-4.5, 1.5-4.0, 2.5-3.5% w/w of polymer, such as about 3% w/w of polymer, e.g. alginate. Accordingly, in some embodiments, the concentration of polymer, e.g. alginate, in the film is about 0.5-5.0% w/w, e.g. about 1.0-4.5, 1.5-4.0, 2.5-3.5% w/w of polymer, such as about 3% w/w of polymer, e.g. alginate.

In some embodiments, the pharmaceutically acceptable soluble film comprises a plasticizer to increase the flexibility of the film. Any suitable plasticizer may be selected from those well-known in the art and may depend on the other materials used to form the film. Representative examples of suitable plasticizers include glycerol, sucrose, sorbitol, maltodextrin, polyethylene glycol, propylene glycol and combinations thereof. In some embodiments, the plasticizer comprises glycerol or propylene glycol. The amount of plasticizer in the film may affect its mucoadhesive properties and this will depend on the materials used to form the film and the selected plasticizer.

In some embodiments, the film solution used to prepare the film comprises about 0.1-5% or about 0.5-5% w/w of plasticizer, e.g. about 1.0-4.5, 1.5-4.0, 2.5-3.5% w/w of plasticizer, such as about 3% w/w of plasticizer, e.g. glycerol or propylene glycol. Accordingly, in some embodiments, the film comprises about 0.1-5% or about 0.5-5% w/w of plasticizer, e.g. about 1.0-4.5, 1.5-4.0, 2.5-3.5% w/w of plasticizer, such as about 3% w/w of plasticizer, e.g. glycerol or propylene glycol.

Thus, in a preferred embodiment, the film solution used to prepare the pharmaceutically acceptable soluble film (e.g. the mucoadhesive pharmaceutically acceptable soluble film) comprises an aqueous solution of alginate polymer comprising 30-40% guluronate and 60-70% mannuronate (e.g. Manucol® LKX, Protanal® CR 8133, Protanal® CR 8233) at a concentration of about 2.5-3.5% w/w and about 2.5-3.5% w/w of glycerol, optionally wherein: (a) a 1% w/v solution of the alginate has a viscosity of 60-170 mPa·s, preferably about 80-130 mPa·s (e.g. Manucol® LKX)(e.g. at about 20° C.); (b) a 2% w/v solution of the alginate (e.g. Protanal® CR 8133) has a viscosity of about 100-300 mPa·s (e.g. at about 20° C.), preferably about 120-250 mPa·s (e.g. at about 20° C.); or (c) a 1.25% w/v solution of the alginate (e.g. Protanal® CR 8233) has a viscosity of about 600-900 mPa·s (e.g. at about 20° C.), preferably about 650-850 mPa·s (e.g. at about 20° C.). Accordingly, in some embodiments, the pharmaceutically acceptable soluble film comprises an alginate polymer comprising 30-40% guluronate and 60-70% mannuronate (e.g. Manucol® LKX, Protanal® CR 8133, Protanal® CR 8233) at a concentration of about 2.5-3.5% w/w and about 2.5-3.5% w/w of glycerol, optionally wherein: (a) a 1% w/v solution of the alginate (e.g. Manucol® LKX) has a viscosity of 60-170 mPa·s (e.g. at about 20° C.), preferably about 80-130 mPa·s (e.g. at about 20° C.); (b) a 2% w/v solution of the alginate (e.g. Protanal® CR 8133) has a viscosity of about 100-300 mPa·s (e.g. at about 20° C.), preferably about 120-250 mPa·s (e.g. at about 20° C.); or (c) a 1.25% w/v solution of the alginate (e.g. Protanal® CR 8233) has a viscosity of about 600-900 mPa·s (e.g. at about 20° C.), preferably about 650-850 mPa·s (e.g. at about 20° C.).

In some embodiments, the film solution used to prepare the pharmaceutically acceptable soluble film (e.g. the mucoadhesive pharmaceutically acceptable soluble film) comprises an aqueous solution of alginate polymer comprising 30-40% guluronate and 60-70% mannuronate (e.g. Manucol® LKX, Protanal® CR 8133, Protanal® CR 8233) at a concentration of about 2.5-3.5% w/w, hyaluronic acid (e.g. hyaluronic acid having an average molecular weight of about 1.5-3.0×10⁶ Da, preferably about 2.0-2.5×10⁶ Da) at a concentration of about 0.25-1.0% and about 0.2-3.0% w/w of glycerol, optionally wherein: (a) a 1% w/v solution of the alginate (e.g. Manucol® LKX) has a viscosity of about 60-170 mPa·s (e.g. at about 20° C.), preferably about 80-130 mPa·s (e.g. at about 20° C.); (b) a 2% w/v solution of the alginate (e.g. Protanal® CR 8133) has a viscosity of about 100-300 mPa·s (e.g. at about 20° C.), preferably about 120-250 mPa·s (e.g. at about 20° C.); or (c) a 1.25% w/v solution of the alginate (e.g. Protanal® CR 8233) has a viscosity of about 600-900 mPa·s (e.g. at about 20° C.), preferably about 650-850 mPa·s (e.g. at about 20° C.).

Accordingly, in some embodiments, the pharmaceutically acceptable soluble film comprises an alginate polymer comprising 30-40% guluronate and 60-70% mannuronate (e.g. Manucol® LKX, Protanal® CR 8133, Protanal® CR 8233) at a concentration of about 2.5-3.5% w/w, hyaluronic acid (e.g. hyaluronic acid having an average molecular weight of about 1.5-3.0×10⁶ Da, preferably about 2.0-2.5×10⁶ Da) at a concentration of about 0.25-1.0% and about 0.2-3.0% w/w of glycerol, optionally wherein: (a) a 1% w/v solution of the alginate (e.g. Manucol® LKX) has a viscosity of about 60-170 mPa·s (e.g. at about 20° C.), preferably about 80-130 mPa·s (e.g. at about 20° C.); (b) a 2% w/v solution of the alginate (e.g. Protanal® CR 8133) has a viscosity of about 100-300 mPa·s (e.g. at about 20° C.), preferably about 120-250 mPa·s (e.g. at about 20° C.); or (c) a 1.25% w/v solution of the alginate (e.g. Protanal® CR 8233) has a viscosity of about 600-900 mPa·s (e.g. at about 20° C.), preferably about 650-850 mPa·s (e.g. at about 20° C.).

In some embodiments, the pharmaceutically acceptable soluble film may comprise other components, e.g. to make the patch of the invention more palatable to the consumer, e.g. a flavour, colour and/or sweetener etc., and/or to improve the shelf-life of the patch, e.g. an antioxidant and/or preservative. Any suitable additional components may be used and conveniently may be added to the film solution described above.

In some embodiments, the mesoporous microparticles loaded with an analgesic agent are added to the film solution such that the microparticles are dispersed and/or distributed in the resultant film. In some embodiments, the pharmaceutically acceptable soluble film contains an analgesic agent (particularly the analgesic agent loaded into the microparticles) in addition to the agent provided by loaded mesoporous microparticles. Alternatively viewed, in some embodiments an analgesic agent (e.g. the analgesic agent loaded into the microparticles) is separately added to the film solution used to produce the mucoadhesive pharmaceutically acceptable soluble film. However, in some embodiments, the pharmaceutically acceptable soluble film does not contain an analgesic agent (particularly the analgesic agent loaded into the microparticles) other than in the form of the loaded mesoporous microparticles. Alternatively viewed, the analgesic agent loaded into the microparticles is not separately added to the film solution used to produce the mucoadhesive pharmaceutically acceptable soluble film.

As noted above, the inventors have determined that mesoporous microparticles are a particularly effective means of providing an analgesic (e.g. topical anaesthetic) agent on or in a mucoadhesive patch for local administration to mucosa (e.g. oral mucosa).

The term “mesoporous” typically refers to materials containing pores with diameters between 2 and 50 nm.

The term “microparticles” typically refers to particles between about 1-1000 μm in size.

Accordingly, mesoporous microparticles are particles having an average (mean) diameter between about 1 and 1000 μm and have pores with diameters between about 2 and 50 nm. Notably however, in the mucoadhesive patch of the invention, it is preferred that the mesoporous particles are sufficiently small to avoid a negative mouth feel, i.e. to avoid being detectable in the mouth by the subject. As noted above, an advantage of the patch of the invention is that the film can dissolve and therefore does not need to be removed from the site of action once the analgesic effect is achieved. As the mesoporous microparticles that form part of the patch will not dissolve, they may disperse in the oral cavity once the film has dissolved and prior to ingestion. Accordingly, it is important that the particles are small enough not to cause discomfort or irritation to the subject. However, there are concerns regarding the uptake of nanoparticles by cells and thus it is preferred that the use of particles in the nanometre range is avoided.

Thus, the mesoporous microparticles for use in the mucoadhesive patch of the invention may have an average (mean) diameter of about 1-50 μm, such as about 1-40 μm, 1-30 μm or 1-20 μm, e.g. an average diameter of less than about 30 μm or 20 μm. In some embodiments, the mesoporous microparticles have an average diameter of about 1-10 μm or 2-5 μm, such as about 1 μm, 2 μm, 3 μm, 4 μm or 5 μm, e.g. about 3 μm.

The mesoporous microparticles for use in the invention typically are monodisperse particles, e.g. particles having a polydispersity index (PDI) (measured as the square of the standard deviation (SD) divided by the mean, where both SD and mean are respective the particle diameters when these are spheres) may range from 0 to 0.1, such as 0 to 0.01. The PDI is ideally less than 0.005.

Alternatively viewed, monodisperse particles are particles with a coefficient of variation of less than 10%, preferably less than 6% and more preferably less than 5%. Coefficient of variation (CV) is determined in percentage as

$$CV = 100 \times standard deviation / mean$$

where mean is the mean particle diameter and standard deviation is the standard deviation in particle size. CV is preferably calculated on the main mode, i.e. by fitting a monomodal distribution curve to the detected particle size distribution. Thus, some particles below or above mode size may be discounted in the calculation which may for example be based on about 90%, more usually about 99% of total particle number (of detectable particles that is). Such a determination of CV is performable on a Field-Effect gun SEM (FE-SEM/FEG-SEM). Polydispersity index can also be determined using such a device.

Mesoporous microparticles may be composed of various substances including silica, calcium carbonate and magnesium carbonate and such particles are commercially available (e.g. Syloid® FP and XDP, Parteck® SLC and Upsalite®). Any suitable mesoporous microparticles with the size characteristics defined above may be used in the mucoadhesive patch of the invention.

Thus, the mesoporous microparticles for use in the mucoadhesive patch of the invention may be selected from mesoporous silica microparticles, mesoporous calcium carbonate microparticles, mesoporous magnesium carbonate microparticles or a combination thereof. In a preferred embodiment, the mesoporous microparticles for use in the mucoadhesive patch of the invention comprise or consist of mesoporous silica microparticles (e.g. Syloid® AL-1 FP or Syloid® 244 FP).

Suitable mesoporous microparticles include particles that can be loaded with a sufficient amount of an analgesic agent as defined herein to enable the release of an effective amount of the analgesic agent under suitable conditions, i.e. upon contact with a mucosal membrane (e.g. oral mucosa). Suitable methods for determining whether mesoporous microparticles can be loaded with, and release, an analgesic agent are described in the Examples.

Mesoporous microparticles may be loaded with an analgesic agent using any suitable means known in the art. In a representative example, the analgesic agent may be provided as a solution (i.e. an analgesic agent dissolved in a suitable solvent, such as a C₁-C₄ alcohol, e.g. lidocaine hydrochloride dissolved in ethanol) and contacted with the mesoporous microparticles (e.g. mesoporous silica microparticles) under conditions suitable for the liquid to be absorbed, e.g. room temperature for at least about 5 minutes. Upon separation of the mesoporous microparticles from the liquid, the microparticles may be treated to allow evaporation of the solvent carrying the analgesic agent to provide the “loaded mesoporous microparticles”, e.g. a dry powder. Any suitable method or treatment may be used to remove the solvent from the mesoporous microparticles, if required.

In some embodiments, the solution comprising the analgesic agent may further contain one or more pharmacologically (or pharmaceutically) acceptable excipients.

The excipient may include any excipients known in the art, for example any carrier or diluent or any other ingredient or agent such as buffer, antioxidant, chelator, flavour, colour, preservative and/or sweetener etc.

The amount of analgesic agent loaded onto the particles needed to provide an effective amount for pain relief will depend on the efficacy of the agent and the amount of particles provided on or in the mucoadhesive patch. In some embodiments, the loaded mesoporous microparticles comprise about 10-60 wt % of the analgesic agent (e.g. lidocaine or a salt thereof), such as about 15-55 wt % or about 20-45 wt %, e.g. about 30-40 wt %, of the analgesic agent. The amount of analgesic agent loaded on the mesoporous microparticles (i.e. in the loaded mesoporous microparticles) may be determined using any suitable means known in the art. In a representative example, the amount of analgesic agent loaded on the mesoporous microparticles is determined by thermogravimetric analysis (TGA).

As the mucoadhesive patch of the invention may be configured to have any size and shape suitable for the utilities described herein, the absolute amount of mesoporous microparticles contained in or on the patch will vary. The efficacy and amount of the analgesic agent in the loaded mesoporous microparticles will also effect the amount of particles required on or in the patch. Accordingly, the amount of mesoporous microparticles contained in or on the patch conveniently may be defined as an amount required to provide an effective amount of analgesic agent to achieve pain relief.

In some embodiments, the amount of mesoporous microparticles contained in or on the patch may be based on the dose of analgesic agent provided per unit of surface area of the patch, i.e. the surface area that will be contacted with the mucosal membrane. Thus, in some embodiments, the mucoadhesive patch contains loaded mesoporous microparticles to provide a dose of analgesic agent of about 0.01-50 mg per cm² of patch, such as about 0.02-50 mg per cm², 0.03-45 mg per cm², 0.04-40 mg per cm², 0.05-35 mg per cm², 0.06-30 mg per cm², 0.07-25 mg per cm², 0.08-20 mg per cm², 0.09-15 mg per cm² or about 0.10-10 mg per cm² of patch, e.g. about 0.1-2.0 mg per cm² of patch.

Alternatively viewed, the mucoadhesive patch provides a total dose of analgesic agent of about 0.01-100 mg, such as about 0.02-100 mg, 0.03-90 mg, 0.04-80 mg, 0.05-70 mg, 0.06-60 mg, 0.07-50 mg, 0.08-40 mg, 0.09-30 g or 0.10-20 mg, e.g. about 0.10-5.0 mg or 0.10-2.0 mg.

The ability of the loaded mesoporous microparticles to release of an effective amount of the analgesic agent under suitable conditions may be determined using any suitable means known in the art. In a representative example, a defined amount of loaded mesoporous microparticles (e.g. 1-5 mg) is added to 5 mL of phosphate-buffered saline (e.g. about pH 6-8, e.g. about pH 7) at room temperature (e.g. 20-25° C.) and the amount of analgesic agent in the solution may be determined from samples taken at various time points, e.g. 1, 2, 5, 10 and 30 minutes. The amount of analgesic agent in the solution may be determined using any suitable means known in the art, such as UV-vis spectroscopy as described in the Examples.

Thus, in some embodiments, the loaded mesoporous microparticles release at least about 80% (e.g. 85%, 90% or more) of the analgesic agent into solution (e.g. phosphate-buffered saline at about pH 6-8, e.g. about pH 7, such as about pH 7.4) at room temperature (e.g. 20-25° C.) in about 10 minutes or less, e.g. 9, 8, 7, 6 or 5 minutes or less. In some embodiments, the loaded mesoporous microparticles release at least about 90% of the analgesic agent into solution (e.g. phosphate-buffered saline at about pH 6-8, e.g. about pH 7, such as about pH 7.4) at room temperature (e.g. 20-25° C.) in about 5 minutes or less, e.g. 4, 3 or 2 minutes or less, such as about 1 minute.

Unexpectedly, it has been determined that release of the analgesic agent from the loaded mesoporous microparticles in the context of the mucoadhesive patch is slower than the release directly from the loaded mesoporous microparticles in solution, particularly when the loaded mesoporous microparticles are distributed or dispersed in a pharmaceutically acceptable soluble film, e.g. the mucoadhesive pharmaceutically acceptable soluble film. This may be particularly advantageous in providing sustained effective pain relief, e.g. for several hours. In this respect, the release of the analgesic agent from the mucoadhesive patch of the invention may be determined using any suitable method. In a representative example, a mucoadhesive patch of defined size (e.g. 20×10 mm) is submerged in 20 mL phosphate-buffered saline (e.g. about pH6-8, e.g. about pH 7, such as about pH 7.4) at room temperature (e.g. 20-25° C.) or body temperature (e.g. about 37° C.), e.g. with gentle stirring (e.g. 10-100 rpm, such as about 50 rpm), and samples are taken at various time points, e.g. 1, 2, 5, 10 and 30 minutes. The amount of analgesic agent in the solution may be determined using any suitable means known in the art, such as UV-vis spectroscopy as described in the Examples. It may be necessary to separate dissolved components in the sample, e.g. by HPLC, to accurately measure the level analgesic agent.

Thus, in some embodiments, a 20×10 mm mucoadhesive patch of the invention releases at least about 30% (e.g. 35%, 40% or more) of the analgesic agent (from the loaded mesoporous microparticles) into solution (e.g. about 20 mL of phosphate-buffered saline at about pH 6-8, e.g. about pH 7, such as about pH 7.4) at body temperature (e.g. about 37° C.) in about 20 minutes or less, e.g. 19, 18, 17, 16 or 15 minutes or less, such as about 10 minutes. Additionally or alternatively, in some embodiments, a 20×10 mm mucoadhesive patch of the invention releases at least about 45% (e.g. 50%, 55% or more) of the analgesic agent (from the loaded mesoporous microparticles) into solution (e.g. about 20 mL of phosphate-buffered saline at about pH 6-8, e.g. about pH 7, such as about pH 7.4) at body temperature (e.g. about 37° C.) in about 40 minutes or less, such as about 35 or 30 minutes.

As noted above, the size and shape of the mucoadhesive patch is not critical to its function. It is simply required that the patch is small enough to adhere to a mucosal membrane in a body cavity as described herein (e.g. the oral cavity) and large enough to provide an effective amount of the analgesic agent at, or in proximity to, the required site of action, e.g. the lesion (e.g. sore, ulcer, wound etc.). In some embodiments, the mucoadhesive patch is large enough to cover completely the required site of action, e.g. the lesion.

Thus, in some embodiments, the mucoadhesive patch is configured to provide a surface area in contact with a mucosal membrane of about 0.5-10 cm², such as about 1-5 cm².

The mucoadhesive patch may be provided in any shape convenient to achieve the utilities defined herein. Thus, the mucoadhesive patch may be a regular or irregular shape. It may have 3 or more sides, e.g. 3-15 sides or it may have a single side, e.g. it may be disc or an irregular shape with rounded sides. In some embodiments, the patch is a strip (e.g. rectangular), disc (e.g. circle, oval, ellipse) or wedge shape. It may have straight and/or curved sides. It may have angular and/or curved corners (vertices).

The term “analgesic agent” refers to agents suitable for local pain relief and therefore includes local and topical anaesthetics, i.e. agents that affect sensation, e.g. that result in a reversible loss of sensation for a limited region of the body without affecting consciousness. Thus, the analgesic agent may be a local anaesthetic agent. Such agents are well-known in the art and any such agent may be used in the mucoadhesive patch of the invention. Examples of suitable analgesic (e.g. local anaesthetic) agents for use in the mucoadhesive patch of the invention (i.e. to be loaded in the mesoporous microparticles described herein) include lidocaine, benzydamine (also known as Tantum Verde, Difflam and Septabene), benzocaine, butamben, dibucaine, oxybuprocaine, pramoxine, proxymetacaine (also known as proparacaine), tetracaine (also known as amethocaine), articaine, bupivacaine (also known as Marcaine), carbocaine (also known as Mepivacaine), phenol oropharyngeal (also known as Cepastat, Ulcerease and Chloraseptic) and prilocaine (also known as Citanest). References to any analgesic (e.g. local anaesthetic) agent defined herein includes its pharmaceutically acceptable salts.

Thus, the analgesic (e.g. local anaesthetic) agent may be selected from any one or more of lidocaine, benzydamine, benzocaine, butamben, dibucaine, oxybuprocaine, pramoxine, proxymetacaine, tetracaine, articaine, bupivacaine, carbocaine, phenol oropharyngeal and prilocaine and pharmaceutically acceptable salts thereof, or a combination thereof. In some embodiments, the analgesic agent is lidocaine, benzydamine or a pharmaceutically acceptable salt thereof, or a combination thereof.

Pharmaceutically acceptable salts include pharmaceutical acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylat and benzene sulfonic acid salts.

Preferred salts include inorganic acid addition salts, particularly hydrochloride. Thus, in some embodiments, the analgesic agent is lidocaine hydrochloride and/or benzydamine hydrochloride.

The loaded mesoporous microparticles described herein (i.e. the mesoporous microparticles loaded with an analgesic agent as defined above) may be incorporated into the mucoadhesive patch in any suitable configuration using any suitable means known in the art.

In some embodiments, the loaded mesoporous microparticles are adhered to a surface of the pharmaceutically acceptable soluble film, e.g. the surface to be contacted with the mucosal membrane. Alternatively viewed, in some embodiments, the mucoadhesive patch comprises mesoporous microparticles loaded with an analgesic agent adhered to a surface of the pharmaceutically acceptable soluble film.

As noted above, the mucoadhesive patch may comprise a plurality of layers of pharmaceutically acceptable soluble film. Thus, in some embodiments, the loaded mesoporous microparticles are interposed between layers of pharmaceutically acceptable soluble film, e.g. between two or more layers. In this embodiment, the loaded mesoporous microparticles are not adhered the surface to be contacted with the mucosal membrane. Alternatively viewed, in some embodiments, the mucoadhesive patch comprises mesoporous microparticles loaded with an analgesic agent interposed between layers of pharmaceutically acceptable soluble film. The loaded mesoporous microparticles may be adhered to the internal surface of one or both layers of pharmaceutically acceptable soluble film.

In some embodiments, the loaded mesoporous microparticles are dispersed in the pharmaceutically acceptable soluble film. Alternatively viewed, in some embodiments, the mucoadhesive patch comprises mesoporous microparticles loaded with an analgesic agent dispersed in a pharmaceutically acceptable soluble film.

For the avoidance of doubt, the loaded mesoporous microparticles are not dispersed in a liquid medium within the patch (e.g. between two layers of pharmaceutically acceptable soluble film) or on the surface of a pharmaceutically acceptable soluble film, e.g. the mucoadhesive pharmaceutically acceptable soluble film.

When the mucoadhesive patch of the invention comprises a plurality of layers of pharmaceutically acceptable soluble film, it will be understood that only the layer contacted with the mucosal membrane needs to have the mucoadhesive properties defined above. Thus, for instance, when the mucoadhesive patch comprises mesoporous microparticles loaded with an analgesic agent interposed between layers of pharmaceutically acceptable soluble film, the layer opposed to the layer of mucoadhesive pharmaceutically acceptable soluble film (e.g. alginate film as defined above) may be non-mucoadhesive or less mucoadhesive than the layer of mucoadhesive pharmaceutically acceptable soluble film.

Accordingly, in some embodiments, the mucoadhesive patch of the invention comprises a plurality of layers of pharmaceutically acceptable soluble film, wherein two or more layers are composed of different substances. In a particular embodiment, the mucoadhesive patch of the invention may comprise a first layer comprising a mucoadhesive pharmaceutically acceptable soluble film and a second layer comprising a pharmaceutically acceptable soluble film that is less mucoadhesive than the first layer, e.g. non-mucoadhesive. In some embodiments, the loaded mesoporous microparticles are interposed between the first and second layers.

In some embodiments, the loaded mesoporous microparticles are adhered to the surface of the first layer to be contacted with the mucosal membrane, or distributed or dispersed with the first layer to be contacted with the mucosal membrane. Thus, in some embodiments, the second layer may be viewed as a backing layer, e.g. to prevent or minimise adhesion of the patch to other mucosal membranes in proximity to the required site of action (e.g. mucosal membranes opposite the site of action).

Thus, in some embodiments, the mucoadhesive patch of the invention comprises a non-mucoadhesive surface opposed to a mucoadhesive surface. For instance, the mucoadhesive patch of the invention may comprise mesoporous microparticles loaded with an analgesic agent interposed between a non-mucoadhesive pharmaceutically acceptable soluble film and a mucoadhesive pharmaceutically acceptable soluble film. Alternatively viewed, the mucoadhesive patch of the invention may comprise a non-mucoadhesive layer (e.g. film) on the surface of the mucoadhesive patch opposed to the surface that will adhere to a mucosal membrane (e.g. the mucoadhesive pharmaceutically acceptable film in which the loaded mesoporous microparticles are distributed or dispersed).

A “non-mucoadhesive” surface or pharmaceutically acceptable soluble film refers to surface (e.g. coating) or film that does not substantially adhere to mucosa upon contact with the mucosa or does so to a lesser degree than a mucoadhesive patch (e.g. film) defined above. Accordingly, a non-mucoadhesive surface or film refers to a surface or film that is able to move (e.g. detach) substantially from the point of contact with the mucosa. Alternatively viewed, a non-mucoadhesive surface or film refers to a surface or film that is readily released from a mucosa following contact, i.e. a surface or film that adheres to mucosa temporarily or transiently.

Thus, in some embodiments, a non-mucoadhesive surface or film has an average pull-off force of less than about 3.0N or about 2.0N, e.g. less than about 1.5N, 1.0N or 0.5N, such as about 0.25N or less, as measured using the method described above and in more detail in the Examples (e.g. wherein the force is an average of about 5 measurements).

In some embodiments, a non-mucoadhesive surface or film has an average work of adhesion of less than about 0.5 Ns, 0.4 Ns, 0.3 Ns, 0.2 Ns, 0.1 Ns or 0.05 Ns, e.g. less than about 0.04 Ns, 0.03 Ns or 0.02 Ns, such as about 0.01 Ns or less, as measured using the method described above and in more detail in the Examples (e.g. wherein the force is an average of about 5 measurements).

In some embodiments, the non-mucoadhesive surface (e.g. second layer or film, e.g. backing layer, e.g. a non-mucoadhesive pharmaceutically acceptable film) of the mucoadhesive patch may have a larger surface area than the mucoadhesive surface (e.g. layer or film). In some embodiments, the non-mucoadhesive surface (e.g. second layer or film, e.g. backing layer) of the mucoadhesive patch may function to minimise the release of the analgesic agent to sites other than the required site of action, e.g. it may have barrier properties to reduce or prevent release of the analgesic agent into the oral cavity.

In some embodiments, the non-mucoadhesive film or surface (e.g. coating) may comprise hydroxypropyl methylcellulose (HPMC). Alternatively viewed, the second layer (e.g. backing layer) of the mucoadhesive patch comprises or consists of a non-mucoadhesive pharmaceutically acceptable soluble film comprising hydroxypropyl methyl cellulose (HPMC) and optionally a plasticizer (e.g. glycerol or propylene glycol) as described above. As noted above, the term “non-mucoadhesive” includes films that are less mucoadhesive than the mucoadhesive pharmaceutically acceptable soluble film used in the patch under the same conditions.

Thus, in the embodiments, the invention provides a mucoadhesive patch comprising two layers, wherein:
a) a first layer comprises a mucoadhesive pharmaceutically acceptable soluble film; and
b) a second layer comprises a pharmaceutically acceptable soluble film that is less mucoadhesive than the mucoadhesive pharmaceutically acceptable soluble film under the same conditions,
wherein a surface of the second layer is available to contact a mucosal membrane when the first layer is adhered to a mucosal membrane.

As shown in the Examples below, it has been surprisingly shown that the mucoadhesivity of a film comprising HPMC can be reduced by adding polyvinylpyrrolidone (PVP) and/or a plasticiser, as described above.

Thus, the non-mucoadhesive layer (e.g. backing layer) of the patch may comprise or consist of a non-mucoadhesive pharmaceutically acceptable soluble film comprising HPMC and polyvinylpyrrolidone (PVP) and optionally a plasticizer (e.g. glycerol or propylene glycol). In some embodiments, the ratio of HPMC and PVP in the non-mucoadhesive pharmaceutically acceptable soluble film (e.g. backing layer) is about 2:1 to about 1:2, preferably about 1:1.

The non-mucoadhesive layer (e.g. backing layer) or the non-mucoadhesive pharmaceutically acceptable soluble film may comprise about 1.0-3.0% HPMC w/w (e.g. about 1.0-2.0 HPMC) and optionally about 1.0-2.0% w/w PVP, preferably about 1.5% w/w HPMC and about 1.5% w/w PVP.

Any suitable form of HPMC may be used to prepare the non-mucoadhesive pharmaceutically acceptable soluble film. In a preferred aspect, the HPMC may comprise about 19-24% methoxy groups and about 4-12% hydroxypropoxy groups, optionally wherein a 2% w/v solution of the HPMC has a viscosity of 3000-6000 mPa·s at about 20° C., preferably about 3500-4500 mPa·s at about 20° C.

As noted above, the viscosity of a solution (e.g. a polymer solution, such as an alginate solution) may be determined using any suitable means known in the art, e.g. using a viscometer such as a capillary kinematic viscometer or a rotational viscometer. In some embodiments, the viscosity of HPMC may be determined using a rotational viscometer.

Any suitable form of PVP may be included in the non-mucoadhesive pharmaceutically acceptable soluble film. The PVP may be a low molecular weight PVP, e.g. with an average molecular weight of about 5000 Da to about 15000 Da, or it may be a high molecular weight PVP, e.g. with an average molecular weight of about 50 kDa to about 500 kDa.

In some preferred embodiments, the PVP may have an average molecular weight of about 7500 Da to about 12500 Da, preferably about 9000 Da to about 11000 Da, such as about 10000 Da.

In some other preferred embodiments, the PVP may have an average molecular weight of about 250 kDa to about 450 kDa, preferably about 325 kDa to about 375 kDa, such as about 360 kDa.

The loaded mesoporous microparticles may be incorporated in the mucoadhesive patch of the invention, e.g. adhered to a surface of a pharmaceutically acceptable soluble film, using any suitable means.

The loaded mesoporous microparticles may be distributed in or on a pharmaceutically acceptable soluble film in any suitable configuration. In some embodiments, the loaded mesoporous microparticles may be distributed substantially uniformly (homogeneously), e.g. on the surface of a pharmaceutically acceptable soluble film. In some embodiments, the loaded mesoporous microparticles may be distributed or dispersed substantially uniformly (homogeneously) within the pharmaceutically acceptable soluble film (e.g. within the mucoadhesive pharmaceutically acceptable soluble film. In some embodiments, the loaded mesoporous microparticles may be concentrated at one or more areas on or in the pharmaceutically acceptable soluble film. For instance, the loaded mesoporous microparticles may be located at a central portion or area of a pharmaceutically acceptable soluble film.

Thus, in a further aspect, the invention provides a method for producing a mucoadhesive patch of the invention comprising:
(a) providing: (i) a pharmaceutically acceptable soluble film; and (ii) mesoporous microparticles loaded with an analgesic agent;
(b)(i) adhering the mesoporous microparticles loaded with an analgesic agent to a surface of the film of (a)(i); and/or (ii) encapsulating (e.g. sandwiching) the mesoporous microparticles loaded with an analgesic agent between the film of (a)(i) and a second pharmaceutically acceptable soluble film.

As noted above, the second pharmaceutically acceptable soluble film may be the same as the film defined in (a)(i), i.e. the first film. However, in some embodiments, the second pharmaceutically acceptable soluble film may be composed of a different substance, e.g. it may be a non-mucoadhesive pharmaceutically acceptable soluble film as defined above.

In some embodiments, a mucoadhesive patch comprising loaded mesoporous particles as defined herein interposed between layers of pharmaceutically acceptable soluble film may be produced by adhering the loaded mesoporous particles to a surface of the first or second pharmaceutically acceptable soluble film and subsequently adhering the other pharmaceutically acceptable soluble film to the film to which the loaded mesoporous particles are adhered. In some embodiments, the mucoadhesive patch comprising loaded mesoporous particles as defined herein interposed between layers of pharmaceutically acceptable soluble film may be produced by adhering the loaded mesoporous particles to a surface of the first and second pharmaceutically acceptable soluble film and subsequently adhering the loaded mesoporous particle coated surfaces together.

Advantageously and unexpectedly, the inventors have determined that the loaded mesoporous microparticles may be adhered to the surface of pharmaceutically acceptable soluble film using aqueous vapour (water vapour), thereby avoiding the use of adhesive compounds.

Thus, in some embodiments, the step of adhering the mesoporous microparticles loaded with an analgesic agent to a surface of a pharmaceutically acceptable soluble film comprises exposing the surface to aqueous vapour and contacting the exposed surface with the mesoporous microparticles loaded with an analgesic agent.

While adhering the loaded mesoporous microparticles to a surface a pharmaceutically acceptable soluble film may be achieved using the method described above, it has been determined that it is challenging to produce patches with a consistent amount of loaded mesoporous microparticles when the patches are produced on a large scale. This is problematic because pharmacopeia regulations stipulate that dose variations of a pharmaceutical product should not exceed 15%, i.e. variations in the dose of analgesic agent provided by the patch of the invention should not be greater than 15%.

Accordingly, the inventors sought to develop other method of manufacture and determined that incorporating the loaded mesoporous microparticles in the film solution used to make the pharmaceutically acceptable soluble film not only simplifies the manufacturing process, it also functions to produce patches with improved sustained release properties.

Thus, the invention also provides a method for producing a mucoadhesive patch comprising:
(i) transferring a solution comprising: (a) alginate and optionally hyaluronic acid; and (b) mesoporous microparticles loaded with an analgesic agent, to a substrate or support (e.g. cast or mould) in an amount suitable to prepare a mucoadhesive pharmaceutically acceptable soluble film in which the mesoporous microparticles are distributed or dispersed within the film;
(ii) incubating the substrate or support under conditions to produce a mucoadhesive pharmaceutically acceptable soluble film, thereby producing the mucoadhesive patch; optionally
(iii) transferring a solution comprising hydroxypropyl methyl cellulose (HPMC) and optionally polyvinylpyrrolidone (PVP) to the surface of the mucoadhesive pharmaceutically acceptable soluble film produced in (ii) in an amount suitable to prepare a film comprising HPMC and optionally PVP adhered to the mucoadhesive pharmaceutically acceptable soluble film; and
(iv) incubating the product of (iii) under conditions to suitable for the solution comprising HPMC and optionally PVP to dry, thereby producing the mucoadhesive patch.

In some embodiments, the method comprises a further step of adhering one or more additional layers to the mucoadhesive patch, e.g. to provide a backing layer as described above.

In some embodiments, the method may comprise a further step of coating one or both surfaces of the mucoadhesive patch (i.e. the exposed surfaces of the pharmaceutically acceptable soluble film(s)). For instance, one of the exposed surfaces may be coated to provide a backing layer and/or non-mucoadhesive surface. In some embodiments, one of the exposed surfaces may be coated to provide a further therapeutic agent as defined further below. In some embodiments, one or both of the exposed surfaces may be coated with a substance to make the patch more palatable to the consumer, e.g. a flavour, colour and/or sweetener etc. In some embodiments, one or both of the exposed surfaces may be coated with a substance to improve the shelf-life of the patch, e.g. an antioxidant and/or preservative.

As noted above, the pharmaceutically acceptable soluble film may be prepared in a large sheet, which may be used in the method above. Thus, in some embodiments, the method may comprise a step of cutting or shaping the product of the steps mentioned above to provide the mucoadhesive patch of the invention, i.e. a plurality of patches.

The method may also comprise a step of packaging the mucoadhesive patch(es) obtained from the method steps defined above.

Thus, in a further aspect, the invention provides a packaged product comprising one or a plurality of the mucoadhesive patch of the invention.

Any suitable moisture-resistant packaging may be utilised in the invention, e.g. blister pack, pouch etc. Each patch may be separately packaged in moisture-resistant packaging and a plurality of separately packaged patches may be further packaged in any suitable packaging, e.g. in a cardboard box etc.

In a representative embodiment, the invention provides a mucoadhesive patch comprising a pharmaceutically acceptable soluble alginate film and mesoporous silica microparticles loaded with lidocaine or a pharmaceutically acceptable salt thereof and/or benzydamine or a pharmaceutically acceptable salt thereof.

In the representative embodiment, the mesoporous silica microparticles may be interposed between two layers of a pharmaceutically acceptable soluble alginate film.

In the representative embodiment, the pharmaceutically acceptable soluble alginate film may comprise an alginate polymer comprising 30-40% guluronate and 60-70% mannuronate, optionally at a concentration of about 0.5-5.0% w/w, further optionally wherein a 1% w/v solution of the alginate has a viscosity of 60-170 mPa·s, preferably about 80-130 mPa·s.

In the representative embodiment, the mesoporous silica microparticles may have an average (mean) diameter of about 1-50 μm and optionally comprise about 10-60 wt % of the analgesic agent, i.e. lidocaine or a pharmaceutically acceptable salt thereof and/or benzydamine or a pharmaceutically acceptable salt thereof.

In the representative embodiment, the mucoadhesive patch may contain loaded mesoporous silica microparticles to provide a dose of analgesic agent (i.e. lidocaine or a pharmaceutically acceptable salt thereof and/or benzydamine or a pharmaceutically acceptable salt thereof) of about 0.01-50 mg per cm² of the surface area of the patch.

In the representative embodiment, the pharmaceutically acceptable salt may be the hydrochloride salt.

As discussed above, the mucoadhesive patch of the invention finds particular utility in the provision of local pain relief on mucosal membranes, particularly in the oral cavity, e.g. pain associated with a disease, disorder, condition or trauma of the oral cavity. Thus, the mucoadhesive patch of the invention may be viewed as a mucoadhesive pharmaceutical composition for local administration to a mucosal membrane (e.g. oral mucosa) to effect pain relief.

Accordingly, in one aspect the invention provides a mucoadhesive patch of the invention for use in therapy.

Alternatively viewed, the invention provides a method for treating and/or preventing a disease or condition (e.g. a disease, disorder or condition of the oral cavity) in a subject, which comprises the step of administering a mucoadhesive patch according to the invention to the subject.

From another perspective, the invention provides the use of a pharmaceutically acceptable soluble film as defined herein and mesoporous microparticles loaded with an analgesic agent (i.e. loaded mesoporous microparticles as defined herein) in the manufacture of a mucoadhesive patch for therapy.

In a further aspect, the invention provides a mucoadhesive patch of the invention for use in treating (e.g. relieving) and/or preventing pain, e.g. pain associated with or in proximity to a mucosal membrane, such as oral mucosa.

Alternatively viewed, the invention provides a method for treating (e.g. relieving) and/or preventing pain (e.g. pain associated with or in proximity to a mucosal membrane, such as oral mucosa) in a subject, which comprises the step of administering a mucoadhesive patch according to the invention to the subject (e.g. at or in proximity to the site of pain or expected pain).

From another perspective, the invention provides the use of a pharmaceutically acceptable soluble film as defined herein and mesoporous microparticles loaded with an analgesic agent (i.e. loaded mesoporous microparticles as defined herein) in the manufacture of a mucoadhesive patch for treating (e.g. relieving) and/or preventing pain, e.g. pain associated with or in proximity to a mucosal membrane, such as oral mucosa.

In yet another aspect, the invention provides a mucoadhesive patch of the invention for use in treating a disorder, condition or trauma of the oral cavity, such as lesions of the oral mucosa, e.g. aphthous stomatitis.

Alternatively viewed, the invention provides a method for treating a disorder, condition or trauma of the oral cavity (e.g. a lesion of the oral mucosa, such as aphthous stomatitis) in a subject, which comprises the step of administering a mucoadhesive patch according to the invention to the subject (e.g. at or in proximity to the site of the lesion).

From another perspective, the invention provides the use of a pharmaceutically acceptable soluble film as defined herein and mesoporous microparticles loaded with an analgesic agent (i.e. loaded mesoporous microparticles as defined herein) in the manufacture of a mucoadhesive patch for treating a disorder, condition or trauma of the oral cavity, such as a lesion of the oral mucosa, e.g. aphthous stomatitis.

As defined herein “treating” or “treatment” as used herein refers broadly to any effect or step (or intervention) beneficial in the management of a clinical condition or disorder. Treatment therefore may refer to reducing, alleviating, ameliorating, slowing the development of, or eliminating one or more symptoms of the disorder which is being treated, relative to the symptoms prior to treatment, or in any way improving the clinical status of the subject. A treatment may include any clinical step or intervention which contributes to, or is a part of, a treatment programme or regimen. In particular, said treatment may comprise reduction in the pain associated with the disorder (e.g. aphthous stomatitis) being treated.

A treatment may include delaying, limiting, reducing or preventing the onset of one or more symptoms (e.g. pain) of the disorder, for example relative to the disorder or symptom prior to the treatment. Thus, treatment explicitly includes both absolute prevention of occurrence or development of symptom of the disorder, and any delay in the development of the disorder or symptom (e.g. pain), or reduction or limitation on the development or progression of the disorder or symptom (e.g. pain, i.e. relieving pain).

Treatment according to the invention thus includes reducing, alleviating eliminating or preventing pain associated with the disorder (e.g. aphthous stomatitis). The term “treatment” does not necessarily imply complete abolition or elimination of pain.

The “subject” or “patient” is an animal (i.e. any human or non-human animal), preferably a mammal, most preferably a human.

In preferred embodiments, the mucoadhesive patch is for administration to oral mucosa as defined above. The mucoadhesive patch may be applied to the site of pain (e.g. directly to a lesion) or in proximity to site of pain, i.e. close enough to the site of pain to provide effective pain relief at the site of pain.

The mucoadhesive patch may find utility in the treatment of any disorder, disease, condition or trauma of a mucosal membrane, particularly an oral mucosal membrane, associated with pain. Such disorders of the oral cavity include lesions (e.g. ulcers or sores) caused by mechanical, chemical, radiation or idiopathic trauma.

The term “lesion” refers to a region of tissue that has suffered damage through injury or disease. Thus, the term lesion includes any sore, ulcer or wound (e.g. abrasion, scratch, cut, tear, puncture etc.) on a mucosal membrane (e.g. oral mucosa), particularly an aphthous ulcer.

The mucoadhesive patch may also find utility in providing pain relief associated with dentition, e.g. in tooth eruption. In some aspects, the mucoadhesive patch may be administered prior to a dental procedure, i.e. to prevent pain associated with the procedure.

In some embodiments, the disorder is aphthous stomatitis, which is characterized by the repeated formation of benign and non-contagious mouth ulcers (aphthae). In some embodiments, the mucoadhesive patch is used to treat ulcers on the non-keratinizing epithelial surfaces in the mouth.

In some embodiments, the mucoadhesive patch is used to treat minor aphthous ulceration (MiAU) (also known minor recurrent aphthous stomatitis (MiRAS)), which refers to ulcers with a diameter of less 10 mm in diameter (typically about 2-3 mm),

In some embodiments, the mucoadhesive patch is used to treat major aphthous ulceration (MaAU) (also known as major recurrent aphthous stomatitis (MaRAS)), which refers to ulcers that have a diameter of 10 mm or more and the ulceration is deeper.

In some embodiments, the mucoadhesive patch is used to treat herpetiform ulcers (also known as stomatitis herpetiformis or herpes-like ulcerations), which refer to a subtype of aphthous stomatitis comprising lesions that resemble a primary infection with herpes simplex virus (primary herpetic gingivostomatitis).

As an episode of aphthous stomatitis may involve a plurality of lesions, i.e. ulcers, the mucoadhesive patch may be used to treat multiple lesions. Alternatively viewed, the mucoadhesive patch may be used to treat multiple aphthous stomatitis.

Thus, the terms “site of action” or “site of pain” refer to areas of the mucosal membrane to be treated that are the cause of pain, e.g. comprising a lesion, or are expected to cause pain, e.g. that are the site of, or in proximity to the site of, a dental procedure or tooth eruption.

As noted above, the mucoadhesive patch of the invention is configured to provide a “therapeutically effective amount” of an analgesic agent, which refers to an amount sufficient to achieve a reduction or decrease in pain. Suitably, a therapeutically effective amount of the analgesic agent is an amount sufficient to induce the desired result without causing a substantial cytotoxic effect in the subject or other negative side effects, such as numbness to other areas which can result in bite injuries and throat, speech and/or swallowing problems.

The effective amount of the analgesic agent useful for eliminating, reducing, alleviating and/or preventing pain will be dependent on the subject being treated, the type and severity of any associated disease, disorder, condition and/or trauma.

“Reducing, relieving or alleviating” pain in a subject may refer to lessening or decreasing the intensity, severity or seriousness of the pain and/or lessening or shortening of the length of time a subject experiences pain, i.e. at the site of action or site of pain. Such reducing, relieving or alleviating need not be absolute to be beneficial to the subject.

“Administering” the mucoadhesive patch of the invention refers to applying the patch to a mucosal membrane at the site of action or site of pain, or in proximity to thereto, with sufficient force and duration to achieve adhesion to the mucosal membrane, e.g. finger pressure for at least 5 seconds. Thus, administration herein refers to both topical and local administration.

It will be understood that the mucoadhesive patch may comprise additional therapeutic agents that are useful in treating the disorders, diseases, conditions and/or trauma mentioned above. The additional therapeutic agents may be incorporated in the patch using any suitable means, e.g. incorporated in the film solution, applied as a coating to the patch or film, incorporated in mesoporous particles adhered to the film.

In some embodiments, the mucoadhesive patch comprises a therapeutic agent for treating aphthous stomatitis. While currently there is no cure for aphthous stomatitis, various agents may be used to reduce or alleviate the symptoms associated with aphthous ulcers. For instance, antiseptics, e.g. antibiotics, may be applied to the ulcers to promote healing, e.g. by preventing infection, and anti-inflammatory agents may be applied to reduce inflammation (which may also reduce or alleviate pain). Moreover, hyaluronic acid recently has been shown to be a promising treatment for aphthous ulcers.

Thus, in some embodiments the mucoadhesive patch comprises a therapeutic agent selected from an anti-inflammatory agent, an antiseptic agent, e.g. an antibiotic, and/or hyaluronic acid.

In some embodiments, the mucoadhesive patch comprises hyaluronic acid in the mucoadhesive pharmaceutically acceptable soluble film as described above, e.g. about 0.25-1.0% w/w, such as about 0.5% w/w hyaluronic acid, preferably wherein the hyaluronic acid has an average molecular weight of about 1.5-3.0×10⁶ Da, preferably about 2.0-2.5×10⁶ Da.

In some embodiments, the anti-inflammatory agent is a corticosteroid, optionally selected from hydrocortisone, beclomethasone, fluocinonide, clobetasol, betamethasone, dexamethasone or a pharmaceutically acceptable salt thereof.

In some embodiments, the antiseptic, e.g. antibiotic, agent is selected from doxycycline, tetracycline, minocycline, chlorhexidine gluconate, triclosan or a pharmaceutically acceptable salt thereof.

Preferred aspects according to the invention are as set out in the Examples in which one or more of the parameters or components used in the Examples may be used as preferred features of the products, uses and methods described hereinbefore.

The invention will now be described in more detail in the following non-limiting Examples with reference to the following drawings in which:

FIG. 1 shows a schematic illustration of the set-up used for measuring mucoadhesion described in Example 6.

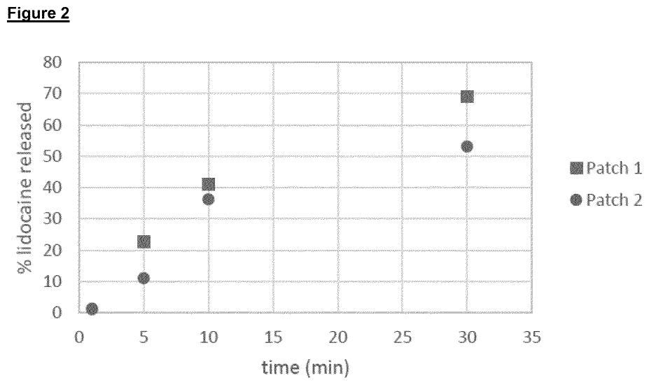

FIG. 2 shows a plot depicting the percentage release of lidocaine from two patches of the invention (patch 1 and patch 2) into PBS as a function of time.

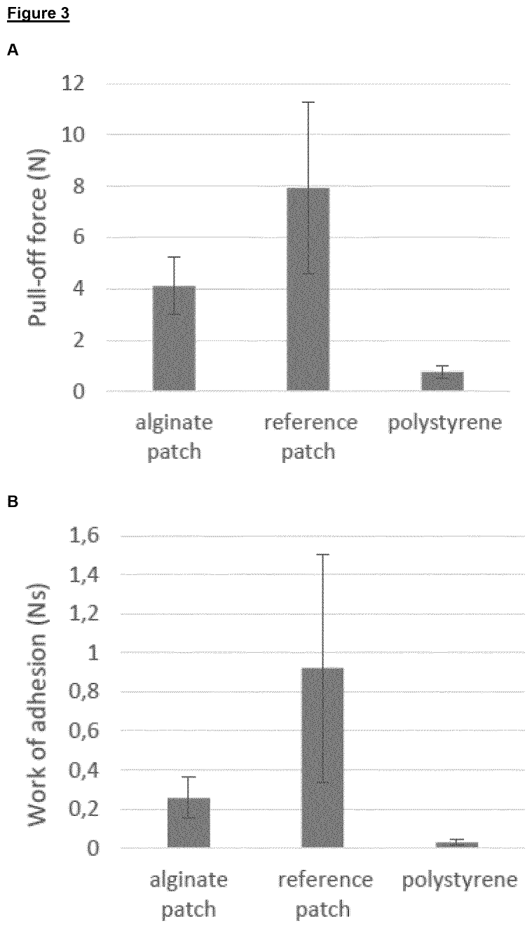

FIG. 3 shows bars charts depicting: (A) pull-off force and (B) work of adhesion for the alginate patch of the invention (alginate patch) in comparison to control surfaces (reference patch and non-adhesive polystyrene).

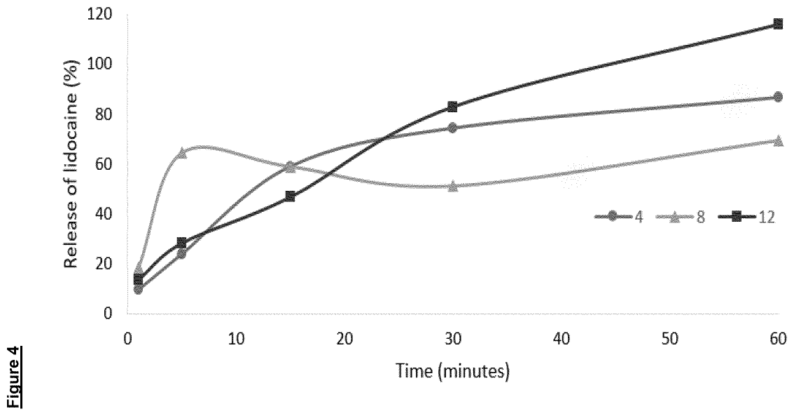

FIG. 4 shows a plot depicting the percentage release of lidocaine from loaded mesoporous microparticles dispersed within the mucoadhesive layer of the patch into PBS as a function of time from three different samples (samples 4, 12 and 8).

### EXAMPLES

### Example 1: Loading Lidocaine into Mesoporous Silica Microparticles (MSMs)

Lidocaine hydrochloride monohydrate was loaded into a pharma grade porous silica (Syloid® 244 FP) with an average particle diameter of about 3 μm producing a dry powder. Loading was achieved by incipient wetness process using lidocaine dissolved in ethanol at a concentration of 50 wt %. The amount of lidocaine loaded into the silica particles was measured by thermogravimetric analysis (TGA). Based on an average from three independent measurements, the amount of lidocaine formed about 35±1 wt % of the powder.

### Example 2: Release of Lidocaine from Mesoporous Silica Microparticles (MSMs)

The release of lidocaine from MSMs into PBS buffer (mimicking saliva) was evaluated at room temperature by UV-vis spectroscopy. Lidocaine loaded silica powder (MSMs) was added to 5 mL phosphate buffered saline (PBS, pH 7.4) and lidocaine concentration in the PBS was measured from the absorbance at the wavelength 263 nm where lidocaine has an absorbance maximum. Samples were taken after 1, 5, 10 and 30 minutes for measurements of the lidocaine concentration in the solution.

The results showed that after 1 minute approximately 90% of the loaded lidocaine had been released from the MSM powder into the solution. That is, MSMs work well as a carrier and do not hinder lidocaine from being released when the MSMs encounter PBS.

### Example 3: Preparation of a Pharmaceutically Acceptable Soluble Film

A stock solution (3.4%) of sodium alginate (Manucol® LKX: medium viscosity pharma grade alginate with a proportion of guluronate/mannuronate of 30-40/60-70) was prepared by dissolving alginate into hot water at 80° C., while stirring using a magnetic stirrer. Stirring was continued until the alginate had dissolved. The stock solution was then used to prepare a film solution by mixing 44 g alginate stock solution with 1.5 g glycerol (added as a plasticizer) and 4.5 g water. A film was cast by pouring 15 g of the solution in a plastic petri dish, and leaving it to dry overnight at 40° C. The resulting film was flexible, and pieces were cut to 2 cm×1 cm for further solubility testing.

The solubility was tested using PBS solution (pH 7.4). The film pieces described above were submerged in 100 mL PBS at room temperature and were left to stand under gentle stirring (about 50 rpm).

After 20 minutes, the film pieces had dissolved completely, indicating that it is suitable for use as the film in an oral patch.

### Example 4: Incorporation of the Lidocaine Loaded MSM Powder onto the Pharmaceutically Acceptable Soluble Alginate Film

Incorporation of the lidocaine loaded MSM powder from Example 1 onto the pharmaceutically acceptable soluble alginate film of Example 3 was not straightforward. As shown in Example 3, the alginate film dissolves in liquids such as water or PBS and, as shown in Example 2, lidocaine is rapidly released from the MSMs in PBS.

It was unexpectedly determined that water vapour from a humidifier could be used to attach the powder on the surface of the alginate film. Patches were prepared by first exposing two pieces of alginate film to water vapour and then contacting the exposed film pieces to loaded MSM silica particles. The MSM-coated surfaces of the film pieces were subsequently pressed together to form a patch comprising loaded MSMs interposed (sandwiched) between the two films.

The resultant patch contained approximately 1.5 mg of loaded MSM powder, which corresponds to approximately 0.5 mg of lidocaine per patch (i.e. about 0.25 mg lidocaine per cm²).

The release of lidocaine from the patches using the solubility test conditions in Example 3 could not be determined by UV-vis spectroscopy as there was interference from the patch ingredients at the wavelengths suitable for detection of lidocaine. The contribution from alginate and glycerol could not be decoupled from the signal form lidocaine. However, the patch was visually dissolved after approximately 20 minutes, in line with the results in Example 3 with the powder-free alginate patch.

### Example 5: Measuring the Release of Lidocaine from the Patch

To determine the rate of release of lidocaine from patches described in Example 4, release measurements were carried out on two lidocaine containing patches and one placebo patch (without lidocaine loaded MSMs). Both lidocaine containing patches were prepared as described in Example 4 each comprising approximately 2.2 mg of loaded MSM powder (i.e. approximately 1.1 mg loaded MSM powder per cm²). This corresponds to approximately 0.77 mg lidocaine per patch.

The patches were held in position between tweezers inside a temperature-controlled vessel containing 20 mL PBS (pH 7.4) at 37° C. with a magnetic stirring bar in the bottom of the vessel set to 50 rpm.

Samples were removed from the vessel for analysis at time points 1, 5, 10, and 30 minutes. The patch was still visible after 10 minutes with some powder remaining undissolved on the patch. After 30 minutes the patch appeared to be completely dissolved by visual inspection.

As the patch ingredients interfere with the UV-vis signal, high pressure liquid chromatography (HPLC) was used to separate the dissolved ingredients and lidocaine, the latter of which was subsequently measured as described in Example 2.

The percentage of lidocaine released from the patch into PBS as a function of time is shown in FIG. 2. After 30 minutes the measured amounts for the two lidocaine loaded patches were 50% and 70%, respectively. This indicates that incorporation of the loaded MSM powder into the alginate patch retards the release of lidocaine, which suggests that the patch is suitable for sustained release of lidocaine and may provide a long duration of pain relief.

### Example 6: Assessment of the Mucoadhesive Properties of the Patch

The mucoadhesivity of the patch described in Example 4 was evaluated by measuring the adhesivity of the patch with texture analyser equipment. A reference patch, (a commercially available vitamin oral patch, Nutrinovate Vitamin Films), and a non-adhesive material, polystyrene, were used as controls.

Glass slides were used as a hydrophilic substrate to mimic oral mucosa. Glass has previously been found to be an appropriate model surface of hydrophilic surface mucosa. The principle of mucoadhesion measurements is shown in the schematic illustration in FIG. 1. The parameters measured are the pull-off force and the work of adhesion.

Glass substrates were cut (approximately 1×1 cm), cleaned and washed carefully before attaching a glass substrate to the texture analyser probe with double-sided adhesive tape. The test patch or control surface was fixed to the base plate of the texture analyser by double-sided adhesive tape. The probe with the glass slide was manually brought to an approximately 1 cm distance above the test patch or control surface, and 15 μL of PBS solution (mimicking saliva) was applied in the middle of the alginate film attached to the instrument's base. Immediately after applying PBS, the test sequence was started, with the probe traveling downwards at a speed of 2 mm/s. Once the probe touched the film, the speed was reduced to 1 mm/s until a contact force of 300 g was reached. The surfaces stayed in contact (300 g contact pressure) for 10 s after which the probe was moved upwards with a speed of 1 mm/s.

The average pull-off force and work of adhesion based on five individual measurements (e.g. 5 patches) and the standard deviation (SD) between them are shown in FIGS. 3A and B, respectively. The results show that the alginate-based patch produced in Example 4 was mucoadhesive with an average pull-off force of 4.1 N and average work of adhesion of 0.3 Ns.

### Example 7: Preparation of Patches

Patches with the formulations shown in Table 1 were manufactured. As described in Example 4, patches were initially prepared by adhering lidocaine loaded MSMs to the surface of soluble films and subsequently adhering the films via their coated surfaces to provide a patch in which the loaded MSMs were sandwiched between the inner and outer layers. However, because this manufacturing approach is not easily achieved in a commercial setting, lidocaine loaded MSMs were then incorporated into the inner (mucoadhesive) layer of the patch as described in Example 8. It was determined that this ensured a comparable dose of the lidocaine loaded MSMs were incorporated into the different patches detailed in Table 1. This approach also allowed for inner (mucoadhesive) and outer (non-mucoadhesive, e.g. backing) layers to be moulded directly together.

Thus, the patches described in Table 1 were made by preparing a solution containing the components of the inner (mucoadhesive) layer, adding and mixing loaded MSMs where indicated (“Lidocaine” in Table 1 below), and pouring 15 g of the solution into a 90 Ø petri dish. The petri dish was transferred to a drying oven/cabinet with circulating air and dried for at least 8 hours or overnight at 40° C. 15 g of a solution containing the components of the outer (non-mucoadhesive/backing) layer was then poured on top of the dried inner layer and again allowed to dry for at least 8 hours or overnight in a drying oven/cabinet at 40° C. The dried two-layered film was then cut into 2 cm×1 cm patches.

TABLE 1
Patches that have been manufactured

| | | | | | Lidocaine |
|---|---|---|---|---|---|
| | Outer | Water | Glycerol | | or |
| Batch | layer | (g) | (g) | Inner layer | Placebo |
| HPMC-A 2% | 1.0 g | 48.9 | 0.1 | 3% Alginate, 0.5% | P |
| | HPMC-A | | | HA | |
| HPMC-A 3% | 1.5 g | 48.4 | 0.1 | 3% Alginate, 0.5% | P |
| | HPMC-A | | | HA | |
| CMC 2% | 1 g CMC | 48.9 | 0.1 | 3% Alginate, 0.5% | P |
| | | | | HA | |
| HPMC-A 1.5%/ | 0.75 g | 47.75 | 0.8 | 3% Alginate, 0.5% | P |
| PVP 1.5% | HPMC-A | | | HA | |
| (3%) Low | 0.75 g | | | | |
| | PVP L | | | | |
| HPMC-A 1.5%/ | 0.75 g | 47.0 | 1.5 | 3% Alginate, 0.5% | P |
| PVP 1.5% | HPMC-A | | | HA | |
| (3%) Med | 0.75 g | | | | |
| | PVP L | | | | |
| HPMC-A 1.5%/ | 0.75 g | 48.4 | 0.1 | 3% Alginate, 0.5% | P |
| PVP High | HPMC-A | | | HA | |
| 1.5% (3%) | 0.75 g | | | | |
| | PVP L | | | | |
| HPMC 2% | 1 g HPMC | 48.9 | 0.1 | 3% Alginate, 0.5% | P |
| Low | 2% | | | HA | |
| HPMC 2% | 1 g HPMC | 48.25 | 0.75 | 3% Alginate, 0.5% | P |
| Med | 2% | | | HA | |
| HPMC 2% | 1 g HPMC | 47.5 | 1.5 | 3% Alginate, 0.5% | P |
| High | 2% | | | HA | |
| HPMC-A 1.5%/ | 0.75 g | 47.0 | 1.5 | 3% Alginate, 0.5% | L |
| PVP 1.5% | HPMC-A | | | HA | |
| (3%) High | 0.75 g | | | | |
| | PVP L | | | | |
| HPMC-A 1.5%/ | 0.75 g | 48.4 | 0.1 | 3% Alginate, 0.5% | L |
| PVP 1.5% | HPMC-A | | | HA | |
| (3%) Low | 0.75 g | | | | |
| | PVP L | | | | |
| HPMC 3% | 1.5 g | 47.9 | 0.1 | 3% Alginate, 0.5% | P |
| Low | HPMC-A | | | HA | |
| HPMC 3% | 1.5 g | 47.75 | 0.75 | 3% Alginate, 0.5% | P |
| Med | HPMC-A | | | HA | |
| HPMC 3% | 1.5 g | 47.0 | 1.5 | 3% Alginate, 0.5% | P |
| High | HPMC-A | | | HA | |
| HPMC-A 1.5%/ | 0.75 g | 48.4 | 0.1 | 3% Alginate | P |
| PVP 1.5% | HPMC-A | | | | |
| (3%) Low | 0.75 g | | | | |
| | PVP L | | | | |
| HPMC-A 1.5%/ | 0.75 g | 48.4 | 0.1 | 3% Alginate | P |
| PVP 1.5% | HPMC-A | | | | |
| (3%) Low | 0.75 g | | | | |
| | PVP H | | | | |
| HPMC/EC | 0.9 g | 48.9 | 0.1 | 3% Alginate, 0.5% | P |
| 90:10 (2%) | HPMC-A | | | HA | |
| Low | 0.1 g EC | | | | |
| HPMC/EC | 0.75 g | 48.9 | 0.1 | 3% Alginate, 0.5% | P |
| 75:25 (2%) | HPMC-A | | | HA | |
| Low | 0.25 g EC | | | | |
| Cross-linked 2 | 0.75 g | 48.4 | 0.1 | 3% Alginate | P |
| mM | HPMC-A | | | 2 mM CaCl₂ | |
| | 0.75 PVP-L | | | | |
| Cross-linked 5 | 0.75 | 48.4 | 0.1 | 3% Alginate | P |
| mM | HPMC-A | | | 5 mM CaCl₂ | |
| | 0.75 PVP-L | | | | |
| HPMC-A 1.5%/ | 0.75 g | 48.4 | 0.1 | 3% proctanol, 0.5% | P |
| PVP High | HPMC-A | | | HA | |
| 1.5% (3%) - | 0.75 g | | | | |
| Proc | PVP L | | | | |

HPMC/HPMC-A: Metolose 90SH-4000 - Hypromellose USP type 2208, 19-24% methoxy/4-12% hydroxypropoxy, nominal viscosity 4000 mPa s.; PVP L = PVP Low viscosity (10 kDa); PVP H = PVP High viscosity (360 kDa); EC = Ethyl cellulose (10 mPa s, hygroscopic); CMC = Carboxymethyl cellulose; Low = 0.1 g glycerol; Medium = 0.75 g glycerol; High = 1.5 g glycerol; Proc = proctanol; Alginate = Protanal ® CR 8233; high viscosity pharma grade alginate; HA = hyaluronic acid (Sodium Hyaluronate, 2.38 × 10⁶ Da) L = inner layer contains lidocaine loaded MSMs; P = non loaded MSMs; MSMs = Porous silica (Syloid 244 FP, Average particle diameter ~3 μm).

### Example 8: Incorporation of the Lidocaine Loaded MSM Powder into a Pharmaceutically Acceptable Soluble Alginate Film

To incorporate lidocaine loaded MSMs into the pharmaceutically acceptable alginate film to serve as the inner layer of the patch, stock solutions of hyaluronic acid (HA) and lidocaine loaded MSMs were produced. First, 0.6 g of HA was added to 20 mL of Milli-Q water (MQ) to make a 3% stock solution. 13 mg of lidocaine was also added to 1.3 mL of MQ to make a 10 mg/mL lidocaine stock solution. 5.57 mg of lidocaine loaded MSMs from Example 1 was then added to 0.225 ml of the lidocaine stock solution. 13.5 g of water was heated to 80° C. and 0.539 g of alginate was slowly stirred into the water. When the solution was fully dissolved, 3.42 g of HA, 0.540 g of glycerol, the lidocaine loaded MSMs and the lidocaine solution were added whilst the solution was stirred. 15 g was poured into a 90 Ø petri dish and dried in the oven at 40° C. overnight. The dried film was cut into 2 cm×1 cm films (16 films in total).

The release of lidocaine from the films was then evaluated as described in Example 5. Four randomised samples of films were chosen and analysed using liquid chromatography (LC); these results are shown in Table 2. The average amount of lidocaine measured across samples was 0.092 mg (relative SD 21.4%).

TABLE 2
The amount of lidocaine measured after dissolution
from 2 cm × 1 cm samples of inner layer.

| Sample | Amount of lidocaine (mg) |
|---|---|
| 4 | 0.110 |
| 14 | 0.076 |
| 18 | 0.108 |
| 21 | 0.074 |

The amount of lidocaine expected to be in each film was theoretically calculated to 0.110 mg (per 2 cm×1 cm film). The difference seen in dose is mainly a result of the film weighing different amounts, resulting in an increased amount of lidocaine in the thicker films. This could be a result of the alginate and HA not being properly dissolved or that the films dry unevenly.

These results demonstrate the successful incorporation the lidocaine loaded MSMs directly into the inner (mucoadhesive) layer of the patch and that the majority of the lidocaine dosage is released from the MSMs.

### Example 9: Measuring the Release of Lidocaine from the Inner Layer of the Patch

To determine the rate of release of lidocaine from the inner layer of the patch, the inner layer was manufactured as described in Examples 7 and 8, and using the materials as outlined in Table 3, with the exception that the total amount of lidocaine was increased to 0.2 mg in each 2 cm×1 cm alginate film.

TABLE 3
The materials and their quantities used to make the inner layer
to determine the rate of lidocaine release from the patch.

| | | | | Lidocaine in | Lidocaine |
|---|---|---|---|---|---|
| Alginate | HA | Glycerol | Water | solution | with MSMs |
| 0.748 g | 4.39 g* | 0.75 g | 19 g | 3.55 mg | 10 mg |

| | | | |
|---|---|---|---|
| 3% | 0.5% | 3% | Total: 7.1 mg |

*Taken from a 3% stock solution.

A 50% thicker inner film was then manufactured to increase the dissolution time of the lidocaine from the film. To make this thicker film, 22.5 g of the solution was poured into a 90 Ø petri dish and dried overnight at 40° C.

The release of lidocaine was measured using a dissolution bath, which was used to heat 20 mL of PBS to 37° C. The film was then lowered onto a stage so that only one side of the film was in contact with the solution, which was maintained at 150 rpm using a magnetic stirrer. 500 μL samples were removed from the solution at the following time points: 1, 5, 15, 30 and 60 minutes. After each sample was collected 500 μL of PBS was added back into the bath to ensure a constant volume of liquid in the heated vessel.

To measure the lidocaine release profile from the inner layer, samples 4, 8 and 12 were randomly selected from the alginate film and their weights are shown in Table 4. The results obtained from measuring lidocaine release from each sample is shown in Table 5.

TABLE 4
The weights of the dried samples of film used to measure
lidocaine release profile of the inner layer

| Sample 4 | Sample 8 | Sample 12 |
|---|---|---|
| 53.9 mg | 39.0 mg | 69.0 mg |

The 500 μL samples were then analysed using liquid chromatography (data not shown). The concentration of lidocaine theoretically expected in a 2 cm×1 cm patch is 0.20 mg. After 60 minutes, sample 4 had released 0.173 mg of lidocaine (equal to 86.5% of the total dose of lidocaine incorporated into the film) as shown in FIG. 4. At this same time point, sample 12 had released 0.2315 mg of lidocaine (115.8%); this excess reflects the larger weight of the patch. Sample 12 weighs 22% more than sample 4 and almost double the weight compared to sample 8.

TABLE 5
The concentrations of lidocaine released from each
sample as measured using LC at each time point.

| Time | Sample 4 | Sample 12 | Sample 8 | Release of lidocaine (%) |
|---|---|---|---|---|
| (min) | (mg) | (mg) | (mg) | n = 3 ± SD |
| 1 | 0.0188 | 0.0268 | 0.0368 | 13.7 ± 3.7 |
| 5 | 0.0475 | 0.0561 | 0.1288 | 38.7 ± 18.2 |
| 15 | 0.1177 | 0.0930 | 0.1177 | 54.7 ± 5.8 |
| 30 | 0.1484 | 0.1654 | 0.1022 | 69.3 ± 13.4 |
| 60 | 0.1730 | 0.2315 | 0.1387 | 90.5 ± 19.2 |

TABLE 6
The concentration of lidocaine released from each sample as measured
using LC at each time point, normalised against the weight of the
patch. The concentrations shown as mg lidocaine/mg patch.

| | | | | Release of lidocaine |
|---|---|---|---|---|
| Time | Sample 4 | Sample 12 | Sample 8 | when normalised against |
| (min) | (mg) | (mg) | (mg) | weight (%) n = 3 ± SD |
| 1 | 0.000349 | 0.000388 | 0.001054 | 14.8 ± 8 |
| 5 | 0.000881 | 0.000813 | 0.003691 | 44.5 ± 33 |
| 15 | 0.002184 | 0.001347 | 0.003372 | 57.1 ± 21 |
| 30 | 0.002752 | 0.002396 | 0.002927 | 66.8 ± 5 |
| 60 | 0.00321 | 0.003355 | 0.003973 | 87.1 ± 8 |

Table 6 presents the concentration of lidocaine released from each sample of patch at each time point normalised to the weight of the patch. Taking into account the estimated average weight of a patch (49.6 mg, mean value of 14 different patches), 80%, 98% and 83% of the total dose (0.2 mg) has been released from each sample. The higher release value for sample 8 (98%), was the result of the patch having a lower weight, thus being thinner and releasing the lidocaine faster.

FIG. 4 shows the release of lidocaine from the inner layer as a function of time for samples 4, 8, and 12.

These results show that the lidocaine loaded MSMs were successfully incorporated into the alginate film. As 90.5% of the incorporated lidocaine was released from the inner layer after 60 minutes in a dissolution bath, as shown in Table 5, this indicates that most of the lidocaine is released from the MSMs. Furthermore, because approximately 50% of the dose had been released after 30 minutes, this suggests a sustained release profile. Thus, incorporation of the loaded lidocaine MSM powder into the inner (mucoadhesive) layer of the patch retards the release of lidocaine, suggesting that the patch is suitable for sustained release of lidocaine and may provide a long duration of pain relief. Furthermore, incorporating lidocaine into the alginate film as opposed to incorporating it onto the film, as outlined in Example 4, is desirable because it makes the lidocaine dosage more controllable.

### Example 10: Assessment of Weight Homogeneity Across the Pharmaceutically Acceptable Soluble Alginate Film

As seen in Example 9, achieving a homogenous weight across the alginate film used to form the inner layer of the patch was not straightforward. This is not due to improper sectioning of the film into 1 cm×2 cm patches but a result of inhomogeneous mixing of the ingredients, including HA, used to make the film.

To try to overcome this, instead of dissolving the alginate and HA powder separately in water and then combining them, the powders were first mixed and added together into water heated to 80° C. After cooling the mixture, glycerol was added and 22.5 g was poured into a 90 Ø petri dish. The petri dish was put on a shaking table for 5 minutes to ensure a homogenous distribution of the solution across the dish. Subsequently, the solution was allowed to dry overnight at 40° C.

The resulting film was cut into 16 2 cm×1 cm patches and the weight of each sample is shown in Table 7. Even after dry mixing the components, a 41% difference was apparent between patches weighing the most and the least. It was hypothesised that more polymer was incorporated into the heavier patches.

TABLE 7
The weights of each sample obtained from one batch
of ingredients used to make an alginate film

| Sample | Weight (mg) |
|---|---|
| 1 | 33.7 |
| 2 | 35.3 |
| 3 | 26.2 |
| 4 | 41.7 |
| 5 | 58.6 |
| 6 | 65.6 |
| 7 | 59.2 |
| 8 | 49.0 |
| 9 | 31.8 |
| 10 | 27.2 |
| 11 | 31.6 |
| 12 | 33.9 |
| 13 | 28.9 |
| 14 | 27.1 |
| 15 | 30.9 |
| 16 | 30.7 |

To further investigate this, mixing times were then evaluated. An alginate gel stock (Alginate 3%, Glycerol 3%, HA 0.5%) was manufactured as outlined in Example 7 and allowed to mix for either 1 hour, 24 hours and 96 hours before being dried in a petri dish as outlined in Example 7. Three randomised 2 cm×1 cm patches were obtained from each of the dried films and weighed. A “Darker” sample was taken from each of the films (indicating increased alginate) and weighed. These results are presented in Table 8.

TABLE 8
The weight of the samples taken from the
three manufactured alginate films.

| | | | | Average | SD | SD (%) of |
|---|---|---|---|---|---|---|
| Film | Film weight (mg) | | | (mg) | (mg) | average |
| Alginate 1 h, 22.5 g | 68.7 | 32.5 | 34.0 | 45.1 | 16.7 | 37% |
| Alginate 24 h, 22.5 g | 35.7 | 57.0 | 35.1 | 42.6 | 10.2 | 24% |
| Alginate 96 h, 22.5 g | 19.9 | 42.0 | 89.3 | 50.4 | 28.9 | 57% |

As shown in Table 8, adjusting the mixing time of the ingredients used to make the film does not improve the homogeneity of the weight of the film.

The drying process of the film was then evaluated. Typically, the alginate films were allowed to dry in cabinet with circulating air. This is common practice in GMP manufacturing. To determine whether the drying process could be adjusted to improve the homogeneity of the film, the following protocols were tested:
Protocol 1:50° C. in an oven using a standard protocol as outlined in previous Examples;
Protocol 2:50° C. in an oven in the presence of an open beaker of saturated NaCl solution to increase humidity and slow the evaporation process;
Protocol 3:50° C. in an oven with the petri dish positioned at the bottom of a large beaker with perforated aluminium foil covering the opening of the beaker. This was to minimise air turbulence over the film during drying.

The weight of each alginate film obtained by using each protocol are presented in Table 9. The inhomogeneity in film weight was reduced when the drying process was slowed using a beaker of saturated NaCl solution (thereby increasing the relative humidity of the air in the oven) and when the air turbulence was minimised. Thus, the drying process is a crucial step in achieving a homogenous film weight and likely for incorporating a homogeneous dose of lidocaine loaded MSMs.

TABLE 9
The weight of the three alginate films obtained
using three separate drying protocols.

| | | | | Average | SD | SD (%) of |
|---|---|---|---|---|---|---|
| Protocol | Film weight (mg) | | | (mg) | (mg) | average |
| 1 | 33.9 | 49.2 | 84.9 | 56.0 | 21.4 | 38% |
| 2 | 31.6 | 36.7 | 59.3 | 42.5 | 12.0 | 28% |
| 3 | 40.0 | 60.4 | 34.7 | 45.0 | 11.1 | 25% |

### Example 10: Assessment of the Parameters Affecting the Dissolution and Mucoadhesiveness of the Patch

The parameters affecting the dissolution of the patch as described in Examples 8 and 9 was further evaluated. Firstly, the thickness of the film was increased to increase dissolution time. Three different alginate films were made as previously described in Examples 8 and 9 (but without the addition of lidocaine or HA). To achieve this, 15 g, 22.5 g and 30 g was poured into a petri dish. Dissolution time was then evaluated by placing the films into 20 mL PBS at 37° C.; each film was monitored until disintegration. The 15 g film achieved dissolution first (by 15-20 minutes) while the other two films weighing 22.3 g and 30 g achieved similar dissolution times of 25-30 minutes.

To reduce brittleness, either 0.1 g or 1 g of glycerol was added to the film weighing 22.5 g, the latter of which resulted in a more flexible film.

Crosslinking the alginate polymers by using CaCl₂) was also tested to evaluate the effect of this on film dissolution time. Either 2 mM or 5 mM CaCl₂) was added to a mixture of alginate, water, and glycerol. The mixture was left to stir overnight to facilitate crosslinking. The mixture was then poured into a 90 Ø petri dish and dried overnight at 40° C.

The outer layer of the patch should be less mucoadhesive than the inner layer, which was hypothesised to help achieve a dissolution time of approximately 60 minutes for the whole patch. The polymer used for the outer layer should be water soluble so that the outer layer has low mucoadhesion and dissolves slowly over time.

To evaluate the effect of the outer layer on dissolution time, HPMC, CMC, HPMC/PVP outer layers were selected. To manufacture a HPMC outer layer, water was heated to 80° C. and HPMC powder was stirred in until dispersed. After the mixture was cooled in an ice bath, glycerol was added. To manufacture a HPMC/EC based outer layer, HPMC powder was added to preheated water during stirring while EC was dissolved in ethanol. Before the HPMC mixture was cooled down, the EC/ethanol solution was added to the mixture. All of these outer layers were dried overnight at 40° C.

To evaluate the dissolution times of these manufactured outer layers, 20 mL of PBS was added to the dissolution bath and heated to 37° C. The film was lowered onto a stage so that only one side of the film was in contact with the solution and a magnetic stirrer was used to stir the solution at 150 rpm. Each film was monitored until dissolution had been achieved and the results of these experiments are shown in Table 10.

TABLE 10
The ingredients used to make film outer (non-mucoadhesive/backing)
layers and the dissolution time achieved by each.

| | | | | Dissolution |
|---|---|---|---|---|
| Batch | Polymer | Water (g) | Glycerol (g) | time (min) |
| HPMC-A 2% | 1 g HPMC-A | 48.9 | 0.1 | 25-30 |
| HPMC-A 3% | 1.5 g HPMC-A | 48.4 | 0.1 | 55 |
| HPMC-A 4% | 2 g HPMC-A | 47.9 | 0.1 | 80 |
| CMC 2% | 1 g CMC | 48.9 | 0.1 | 20 |
| HPMC-A/PVP | 0.5 g HPMC-A | 48.9 | 0.1 | 30-35 |
| Low 1:1 (2%) | 0.5 g PVP L | | | |
| HPMC-A/PVP | 0.5 g HPMC-A | 48.9 | 0.1 | 30 |
| High 1:1 (2%) | 0.5 g PVP H | | | |
| HPMC-A/PVP | 0.75 g HPMC-A | 48.4 | 0.1 | 40-45 |
| Low 1:1 (3%) | 0.75 g PVP L | | | |
| HPMC-A/PVP | 0.75 g HPMC-A | 48.4 | 0.1 | 45 |
| High 1:1(3%) | 0.75 g PVP H | | | |

HPMC/HPMC-A: Metolose 90SH-4000 - Hypromellose USP type 2208, 19-24% methoxy/4-12% hydroxypropoxy, nominal viscosity 4000 mPa s.; PVP L = PVP Low viscosity (10 kDa); PVP H = PVP High viscosity (360 kDa); CMC = Carboxymethyl cellulose.

Table 10 shows that a higher concentration of polymers in the outer layer resulted in an increased dissolution time.

The mucoadhesivity of the films was then evaluated by measuring their adhesivity using the texture analyser equipment as detailed in Example 4 and as shown in FIG. 1.

An outer layer of HPMC-A/PVP Low 1:1 3% was considered to provide suitable non-mucoadhesive properties for the patch, followed by HPMC-A 2% and HPMC-A 3%. Previous experiments have shown that an increase in glycerol decreases the mucoadhesion of the film. Thus, three different glycerol amounts were tested for a several film types including HPMC 2%, HPMC 3% and HPMC/PVP 1:1 3%. The compositions of these prepared films are presented in Table 11.

TABLE 11
The compositions of the various outer layers
manufactured to determine patch mucoadhesion.

| | HPMC- | PVP | | | | |
|---|---|---|---|---|---|---|
| | A | LOW | CMC | Water | Glycerol | Propylene |
| Batch | (g) | (g) | (g) | (g) | (g) | glycol |
| HPMC 2% | 1 | | | 48.9 | 0.1 | |
| Low G | | | | | | |
| HPMC 2% | 1 | | | 48.25 | 0.75 | |
| Medium G | | | | | | |
| HPMC 2% | 1 | | | 47.5 | 1.5 | |
| High G | | | | | | |
| HPMC/PVP | 0.75 | 0.75 | | 47.9 | 0.1 | |
| Low G | | | | | | |
| HPMC/PVP | 0.75 | 0.75 | | 47.25 | 0.75 | |
| Medium G | | | | | | |
| HPMC/PVP | 0.75 | 0.75 | | 47 | 1.5 | |
| High G | | | | | | |
| HPMC 3% | 1.5 | | | 48.4 | 0.1 | |
| Low G | | | | | | |
| HPMC 3% | 1.5 | | | 47.25 | 0.75 | |
| Medium G | | | | | | |
| HPMC 3% | 1.5 | | | 47 | 1.5 | |
| High G | | | | | | |
| HPMC 4% | 2 | | | 47.9 | 0.1 | |
| Low G | | | | | | |
| CMC 2% | | | 1 | 48.9 | 0.1 | |
| Low G | | | | | | |
| HPMC 3% | 1.5 | | | | 48.4 | 0.1 |
| Low PG | | | | | | |

HPMC/HPMC-A: Metolose 90SH-4000 - Hypromellose USP type 2208, 19-24% methoxy/4-12% hydroxypropoxy, nominal viscosity 4000 mPa s.; PVP L = PVP Low viscosity (10 kDa); Low G = 0.1 g glycerol; Medium G = 0.75 g glycerol; High G = 1.5 g glycerol; Low PG = 0.1 g propylene glycol.

The mucoadhesive properties of each outer layer was measured as described in Example 6 and as illustrated in FIG. 2. The outer layer showed a tendency for mucoadhesiveness that remained lower than that previously measured and lower than the inner layer of the patch. Patches were manufactured by varying the softener (plasticiser) between glycerol and 1,2-propylene glycol. Patches were also manufactured using 3% HPMC (K4M) and 0.5% softener (glycerol or propylene glycol) and the weight of each film was noted to monitor whether any variation in film weight is due to the choice of polymer. These results are presented in Table 12.

TABLE 12
Randomised samples selected from different film types to
evaluate mucoadhesivness and the weight of each film.

| | | | | Average | SD | SD (%) of |
|---|---|---|---|---|---|---|
| Film | Film weight (mg) | | | (mg) | (mg) | average |
| HPMC glycerol, 18 g | 8.8 | 10.7 | 27.9 | 15.8 | 8.6 | 54% |
| HPMC propylene | 33.5 | 11.1 | 11.4 | 18.7 | 10.5 | 56% |
| glycol, 18 g | | | | | | |

The films were analysed for mucoadhesion and the results of these analyses are presented in Table 13 and show that the use of propylene glycol as a softener decreased the mucoadhesion of the outer layer.

To further study the effect of softener and the addition of other polymers, HPMC films with and without glycerol were manufactured together with HPMC gels with PVP (polyvinylpyrrolidone). Although the PVP films were difficult to measure with the selected method, the results obtained indicate that a film with a high concentration of HPMC and glycerol is less mucoadhesive than HPMC mixed with PVP (Table 13). However, all films tested displayed suitable properties for use in the patch of the invention.

TABLE 13
The mucoadhesion properties of different film types determined
using the texture analyser as shown in FIG. 1.

| | Pull-off Force | | Work of adhesion | |
|---|---|---|---|---|
| | (N) | SD | (Ns) | SD |
| HPMC 2% low | 4.78 | 0.71 | −28.8 | 0.12 |
| HPMC 2% medium | 4.53 | 0.57 | 0.45 | N/A |
| HPMC 2% high | 2.53 | 1.74 | 0.18 | 0.16 |
| HPMC 3% low | 3.93 | 1.83 | 0.07 | N/A |
| HPMC 3% medium | 4.43 | 0.4 | −28.38 | 0.67 |
| HPMC 3% high | 1.1 | 1.89 | −9.63 | 16.69 |
| HPMC PVP low | 2.26 | 1.18 | 0.88 | 0.72 |
| HPMC PVP medium | 4.685 | 0.015 | 0.655 | 0.145 |
| HPMC PVP high | 4.17 | NA | 0.32 | N/A |

1. A mucoadhesive patch comprising a pharmaceutically acceptable soluble film and mesoporous microparticles loaded with an analgesic agent.

2. The mucoadhesive patch of claim 1, wherein the analgesic agent is lidocaine or a pharmaceutically acceptable salt thereof and/or benzydamine or a pharmaceutically acceptable salt thereof.

3. The mucoadhesive patch of claim 1 or 2, wherein the analgesic agent is lidocaine hydrochloride and/or benzydamine hydrochloride.

4. The mucoadhesive patch of any one of claims 1 to 3, wherein the pharmaceutically acceptable soluble film comprises alginate.

5. The mucoadhesive patch of any one of claims 1 to 4, wherein the mesoporous microparticles have an average particle diameter of about 1-50 μm, optionally about 1-30 μm or 1-20 μm.

6. The mucoadhesive patch of any one of claims 1 to 5, wherein the mesoporous microparticles have an average particle diameter of about 1-10 μm, preferably about 2-5 μm.

7. The mucoadhesive patch of any one of claims 1 to 6, wherein the mesoporous microparticles are mesoporous silica microparticles, mesoporous calcium carbonate microparticles or mesoporous magnesium carbonate microparticles or a combination thereof.

8. The mucoadhesive patch of any one of claims 1 to 7, wherein the mesoporous microparticles are mesoporous silica microparticles.

9. The mucoadhesive patch of any one of claims 1 to 8, wherein the mesoporous microparticles loaded with an analgesic agent comprise about 10-60 wt % of the analgesic agent, optionally about 20-45 wt % of the analgesic agent.

10. The mucoadhesive patch of any one of claims 1 to 9, wherein the mucoadhesive patch contains mesoporous microparticles loaded with an analgesic agent to provide a dose of analgesic agent of about 0.01-50 mg per cm² of patch, optionally about 0.1-10 mg per cm² of patch or 0.1-2 mg per cm² of patch.

11. The mucoadhesive patch of any one of claims 1 to 10, wherein the mucoadhesive patch provides a total dose of analgesic agent of about 0.02-100 mg, optionally about 0.10-20 mg or 0.10-5.0 mg.

12. The mucoadhesive patch of any one of claims 1 to 11, wherein the mucoadhesive patch is configured to provide a surface area in contact with a mucosal membrane of about 0.5-10 cm², optionally about 1-5 cm².

13. The mucoadhesive patch of any one of claims 1 to 12, wherein the mesoporous microparticles loaded with an analgesic agent are adhered to a surface of the mucoadhesive patch.

14. The mucoadhesive patch of any one of claims 1 to 13, wherein the mesoporous microparticles loaded with an analgesic agent are interposed between layers of pharmaceutically acceptable soluble film.

15. The mucoadhesive patch of any one of claims 1 to 12, wherein the mesoporous microparticles loaded with an analgesic agent are distributed or dispersed in the pharmaceutically acceptable soluble film.

16. The mucoadhesive patch of any one of claims 1 to 15, wherein the mucoadhesive patch comprises a non-mucoadhesive surface opposed to a mucoadhesive surface.

17. The mucoadhesive patch of any one of claims 1 to 16, wherein the mucoadhesive patch comprises a non-mucoadhesive layer on the surface of the mucoadhesive patch opposed to the surface that will adhere to a mucosal membrane.

18. The mucoadhesive patch of any one of claims 1 to 17, wherein the mucoadhesive patch comprises a therapeutic agent.

19. The mucoadhesive patch of claim 18, wherein the therapeutic agent is for treating aphthous stomatitis.

20. The mucoadhesive patch of claim 18 or 19, wherein the therapeutic agent is an anti-inflammatory agent, an antiseptic agent, e.g. an antibiotic, and/or hyaluronic acid.

21. A mucoadhesive patch comprising two layers, wherein:
a) a first layer comprises a mucoadhesive pharmaceutically acceptable soluble film; and
b) a second layer comprises a non-mucoadhesive pharmaceutically acceptable soluble film,
wherein a surface of the second layer is available to contact a mucosal membrane when the first layer is adhered to a mucosal membrane.

22. The mucoadhesive patch of claim 21, wherein the first layer and second layer are adhered to each other directly.

23. The mucoadhesive patch of any one of claims 1 to 22, wherein a patch having a surface area of 200 mm² dissolves completely in 100 mL phosphate-buffered saline, pH 7.4 at room temperature in about 30-90 minutes.

24. The mucoadhesive patch of any one of claims 1 to 23, wherein the mucoadhesive pharmaceutically acceptable soluble film comprises alginate and optionally hyaluronic acid.

25. The mucoadhesive patch of claim 24, wherein the mucoadhesive pharmaceutically acceptable soluble film comprises an alginate polymer comprising 30-40% guluronate and 60-70% mannuronate, wherein: (a) a 1% w/v solution of the alginate has a viscosity of 60-170 mPa·s at about 20° C., preferably about 80-130 mPa·s at about 20° C.; (b) a 2% w/v solution of the alginate has a viscosity of about 100-300 mPa·s at about 20° C., preferably about 120-250 mPa·s at about 20° C.; or (c) a 1.25% w/v solution of the alginate has a viscosity of about 600-900 mPa·s at about 20° C., preferably about 650-850 mPa·s at about 20° C.

26. The mucoadhesive patch of any one of claims 1 to 25, wherein the mucoadhesive pharmaceutically acceptable soluble film comprises about 2.0-4.0% w/w alginate and optionally about 0.25-1.0% w/w hyaluronic acid.

27. The mucoadhesive patch of any one of claims 1 to 26, wherein the mucoadhesive pharmaceutically acceptable soluble film comprises about 3.0% w/w alginate and about 0.5% w/w hyaluronic acid.

28. The mucoadhesive patch of any one of claims 24 to 27, wherein the hyaluronic acid has an average molecular weight of about 1.5-3.0×10⁶ Da, preferably about 2.0-2.5×10⁶ Da.

29. The mucoadhesive patch of any one of claims 17 to 28, wherein the non-mucoadhesive layer or the non-mucoadhesive pharmaceutically acceptable soluble film comprises hydroxypropyl methyl cellulose (HPMC) and optionally a plasticizer (e.g. glycerol or propylene glycol).

30. The mucoadhesive patch of any one of claims 17 to 29, wherein the non-mucoadhesive layer or the non-mucoadhesive pharmaceutically acceptable soluble film comprises HPMC and polyvinylpyrrolidone (PVP).

31. The mucoadhesive patch of claim 30, wherein the ratio of HPMC:PVP is about 1:1.

32. The mucoadhesive patch of any one of claims 17 to 31, wherein the non-mucoadhesive layer or the non-mucoadhesive pharmaceutically acceptable soluble film comprises about 1.0-3.0% w/w HPMC and optionally about 1.0-2.0% w/w PVP, preferably about 1.5% w/w HPMC and about 1.5% w/w PVP.

33. The mucoadhesive patch of any one of claims 17 to 32, wherein the HPMC comprises about 19-24% methoxy groups and about 4-12% hydroxypropoxy groups, optionally wherein a 2% w/v solution of the HPMC has a viscosity of about 3000-6000 mPa·s at about 20° C., preferably about 3500-4500 mPa·s at about 20° C.

34. The mucoadhesive patch of any one of claims 30 to 33, wherein the PVP has an average molecular weight of about 7500 Da to about 12500 Da, preferably about 9000 Da to about 11000 Da, optionally about 10000 Da.

35. The mucoadhesive patch of any one of claims 30 to 33, wherein the PVP has an average molecular weight of about 250 kDa to about 450 kDa, preferably about 325 kDa to about 375 kDa, optionally about 360 kDa.

36. The mucoadhesive patch of any one of claims 21 to 35, wherein mesoporous microparticles loaded with an analgesic are distributed or dispersed in the mucoadhesive pharmaceutically acceptable soluble film.

37. The mucoadhesive patch of claim 36, wherein:
(i) the analgesic agent is as defined in claim 2 or 3;
(ii) the mesoporous microparticles are as defined in any one of claims 5 to 9;
(iii) the mucoadhesive patch is as defined in any one of claims 10 to 12 or 18 to 20.

38. The mucoadhesive patch of any one of claims 1 to 37 for use in therapy.

39. The mucoadhesive patch of any one of claims 1 to 37 for use in treating and/or preventing pain.

40. The mucoadhesive patch of any one of claims 1 to 37 for use in treating a disorder, condition or trauma of the oral cavity.

41. The mucoadhesive patch of claim 40, wherein the disorder or condition is aphthous stomatitis.

42. A packaged product comprising the mucoadhesive patch of any one of claims 1 to 37.

43. A method for producing a mucoadhesive patch as defined in any one of claims 1 to 14 or 16 to 19 comprising:
(a) providing: (i) a pharmaceutically acceptable soluble film; and (ii) mesoporous microparticles loaded with an analgesic agent;
(b)(i) adhering the mesoporous microparticles loaded with an analgesic agent to a surface of the film of (a)(i); and/or (ii) encapsulating the mesoporous microparticles loaded with an analgesic agent between the film of (a)(i) and a second pharmaceutically acceptable soluble film.

44. The method of claim 43, wherein step (b)(i) comprises exposing the pharmaceutically acceptable soluble film of (a)(i) to aqueous vapour and contacting the pharmaceutically acceptable soluble film with the mesoporous microparticles loaded with an analgesic agent.

45. A method for producing a mucoadhesive patch comprising:
(i) transferring a solution comprising: (a) alginate and optionally hyaluronic acid; and (b) mesoporous microparticles loaded with an analgesic agent, to a substrate or support (e.g. cast or mould) in an amount suitable to prepare a mucoadhesive pharmaceutically acceptable soluble film in which the mesoporous microparticles are distributed or dispersed within the film;
(ii) incubating the substrate or support under conditions to produce a mucoadhesive pharmaceutically acceptable soluble film, thereby producing the mucoadhesive patch; optionally
(iii) transferring a solution comprising hydroxypropyl methyl cellulose (HPMC) and optionally polyvinylpyrrolidone (PVP) to the surface of the mucoadhesive pharmaceutically acceptable soluble film produced in (ii) in an amount suitable to prepare a film comprising HPMC and optionally PVP adhered to the mucoadhesive pharmaceutically acceptable soluble film; and
(iv) incubating the product of (iii) under conditions to suitable for the solution comprising HPMC and optionally PVP to dry, thereby producing the mucoadhesive patch.

46. The method of claim 45, wherein step (ii) comprises incubating the substrate or support at a temperature of about 45-60° C. (e.g. about 50-55° C.) for at least about 8 hours (e.g. about 8-48 hours or about 12-24 hours) to produce said film,
optionally wherein step (ii) is performed: (a) under conditions that minimise air turbulence at the surface of the substrate or support; and/or (b) at a high relative humidity.

47. A method for producing a mucoadhesive patch or mucoadhesive pharmaceutically acceptable soluble film comprising:
(i) transferring a solution comprising alginate and optionally hyaluronic acid to a substrate or support (e.g. cast or mould) in an amount suitable to prepare a film; and
(ii) incubating the substrate or support at a temperature of about 45-60° C. (e.g. about 50-55° C.) for at least about 8 hours (e.g. about 8-48 hours or about 12-24 hours) to produce said film or patch,
wherein step (ii) is performed: (a) under conditions that minimise air turbulence at the surface of the substrate or support; and/or (b) at a high relative humidity.

48. The method of any one of claims 45 to 47, wherein step (ii) is performed at a high relative humidity, optionally wherein the relative humidity is at least about 40%, preferably at least about 45%, 50%, 55%, 60%, 65% or 70%.

49. The method claim 47 or 48, wherein the solution comprising alginate and optionally hyaluronic acid contains mesoporous microparticles loaded with an analgesic agent and is transferred to a substrate or support (e.g. cast or mould) in an amount suitable to prepare a mucoadhesive pharmaceutically acceptable soluble film in which the mesoporous microparticles are distributed or dispersed within the film.

50. The method of any one of claims 47 to 49, wherein the method is for producing a mucoadhesive patch and comprises:
(iii) transferring a solution comprising hydroxypropyl methyl cellulose (HPMC) and optionally polyvinylpyrrolidone (PVP) to the surface of the mucoadhesive pharmaceutically acceptable soluble film in an amount suitable to prepare a film comprising HPMC and optionally PVP adhered to the mucoadhesive pharmaceutically acceptable soluble film; and
(iv) incubating the product of (iii) under conditions to suitable for the solution comprising HPMC and optionally PVP to dry, thereby producing the mucoadhesive patch.

51. The method of any one of claims 45 to 50, wherein:
(i) the solution comprising alginate and optionally hyaluronic acid, comprises an alginate polymer as defined in claim 25 and optionally hyaluronic acid as defined in claim 28;
(ii) the solution comprising alginate and optionally hyaluronic acid, comprises about 2.0-4.0% w/v alginate and optionally about 0.25-1.0% w/v hyaluronic acid, preferably about 3.0% w/v alginate and about 0.5% w/v hyaluronic acid; and/or
(iii) the mesoporous microparticles are as defined in any one of claims 5 to 9.

52. The method of any one of claim 45, 46, 48, 50 or 51, wherein:
(i) the solution comprising hydroxypropyl methyl cellulose (HPMC) and optionally polyvinylpyrrolidone (PVP), comprises HPMC as defined in claim 33 and optionally PVP as defined in claim 34 or 35;
(ii) the solution comprising HPMC and optionally PVP, comprises about 1.0-2.0% w/v HPMC and optionally about 1.0-2.0% w/v PVP, preferably about 1.5% w/v HPMC and about 1.5% w/v PVP;
(iii) the ratio of HPMC:PVP in the solution comprising HPMC and PVP is about 1:1; and/or
(iv) the solution comprising HPMC and optionally PVP contains a plasticizer, preferably glycol or propylene glycol.
